(19) United States
(12) Patent Application Publication
(10) Pub. No.: US 2022/0062474 A1
(43) Pub. Date: Mar. 3, 2022

(54) DISINFECTING MEDICAL IMAGING SYSTEM

(71) Applicant: CANON MEDICAL SYSTEMS CORPORATION, Otawara-shi (JP)

(72) Inventors: Erin ANGEL, Tustin, CA (US); Qingqing LUKAS, Tustin, CA (US); Eugene MENSAH, Tustin, CA (US)

(73) Assignee: CANON MEDICAL SYSTEMS CORPORATION, Otawara-shi (JP)

(21) Appl. No.: 17/405,178

(22) Filed: Aug. 18, 2021

Related U.S. Application Data
(60) Provisional application No. 63/071,079, filed on Aug. 27, 2020.

(51) Int. Cl. A61L 2/10

(52) U.S. Cl. CPC A61L 2/10; A61L 2202/14; A61L 2202/24

(57) ABSTRACT

A disinfecting medical imaging system including medical imaging equipment and a UV light source for disinfecting the medical imaging equipment. The UV light source can include a composite ultraviolet (UV) for performing viral and bacterial disinfection. The disinfection system may be built into the medical imaging equipment or external thereto such as conveyed on a robotic arm.

### CROSS REFERENCE TO CO-PENDING APPLICATION

The present application claims priority under 35 U.S.C. § 119(e) to U.S. Provisional Patent Application No. 63/071,079, flied Aug. 27, 2020, the contents of which are incorporated herein by reference.

### FIELD OF THE INVENTION

Embodiments described herein relate generally to a system and method of disinfecting medical imaging equipment, and in at least one embodiment to a method and system of disinfecting medical imaging equipment using at least one of an integrated UV light source and a robotically controlled UV light source.

### BACKGROUND

UV rays span the electromagnetic radiation spectrum in the wavelength region of 10 nm to 400 nm. The wavelength of UV rays are shorter than visible light but longer than X-ray. UV radiation is present in sunlight and constitutes about 10% of the total electromagnetic radiation output from the sun.

Ultraviolet (UV) light fixtures and mobile devices use powerful, broad spectrum light including: UV-C, UV-B, and anti-bacterial UV-A to optimize their germicidal (germs killing) efficiency. UV-C light targets the DNA and RNA of microbes. DNA and RNA are the genetic materials that make up all living organisms. Thus, UV light impedes the growth, development, functioning and reproduction of microbes upon exposure. UV-C light also produces electromagnetic energy that can destroy the ability of microorganisms to reproduce/replicate. UV-C is traditionally referred to as germicidal UV with the ability to kill bacteria, viruses, mold, and fungus. UV-A and UV-B light cause oxidation of proteins and lipids leading to cell death. Broad band UV lamps have also been shown to inhibit photo-reactivation, which is the process that can result in self-repair of damaged microbes.

UV-C technologies are used as effective food decontamination solutions in reducing mold and bacterial contamination in food production and storage facilities. This helps to enhance product quality and extend product shelf life. UV-C disinfection products include a range of air and surface decontamination systems, and can be used in industry, hospitals, laboratories, institutions, offices and schools to provide a safer and cleaner environment, energy savings and reduced HVAC maintenance costs.

Far field UV-C light is safe for human skin/eyes. Continuous low doses of far-ultraviolet C (far-UVC) light can kill airborne flu viruses without harming human tissues.

Efficient and automated methods of disinfecting medical imaging equipment and surfaces contaminated with various viruses, including the Middle Eastern respiratory syndrome coronavirus (MERSCoV) and Sever Acute Respiratory Syndrome—Coronavirus 2 (SARS-CoV2) that causes COVID-19, may prevent the spread of these virus. It has been shown and reported that automated triple-emitter whole room UV-C disinfection system to inactivate mouse hepatitis virus, strain A59 (MHV-A59), and MERS-CoV viruses on surfaces with a >5 log 10 reduction can be achieved.

Coronaviruses were first identified as the causative agent of the severe acute respiratory syndrome (SARS) in 2002 in China, and the Middle Eastern respiratory syndrome (MERS) in the Middle East in 2012. See (1) [Nie 2003]: Nie Q H Luo X D, Hui W L. Advances in clinical diagnosis and treatment of severe acute respiratory syndrome; World J Gastroenterol 2003; 9:1139-4143; (2) [de Groot 2013]: de Groot R J, Baker S C, Baric R S, et al. Middle East respiratory syndrome coronavirus (MERS-CoV); announcement of the Coronavirus Study Group; J Virol 2013; 5:13-15; and (3) [Zumla 2015]: Zumla A, Hui D S, Perlman S., Middle East respiratory syndrome; Lancet 2015; 386:995-1007.

The first reported case of MERS occurred in Saudi Arabia in 2012 and resulted in 76 deaths. See [Zaki 2012]: Zaki A M, van Boheemen S, Bestebroer T M, Osterhaus A D M E, Fouchier R A M, Isolation of a novel coronavirus from a man with pneumonia in Saudi Arabia; N Engl J Med 2012; 367:1814-1820. Since that time, cases of MERS have continued to occur in both the Middle East as well as in other countries in Europe, Asia, Africa, and North America. A more recent outbreak, involving more than 180 patients, occurred in South Korea. See [Khan: 015]: Khan A, Farooqui A, Guan Y, Kelvin D J; Lessons to learn from MERS-CoV outbreak in South Korea; J Infect Dev Ctries 2015; 9:7-10.

MERS has a reported mortality rate of approximately 36%. See [deGroot 2013], [Zumla 2015], and [Memish 015]: Memish Z A, Al-Tawfiq J A, Alhakeem R F, et al.; Middle East respiratory syndrome coronavirus (MERS-CoV); a cluster analysis with implications for global management of suspected cases; Travel Med Infect Dis 2015; 13:311-314). Human-to-human transmission also has been identified in hospital and household transmission during MERS outbreaks. The ability of coronaviruses to rapidly mutate increases the risk of a large-scale outbreak or epidemic in the future. See [deGroot 2013].

### SUMMARY

Current imaging equipment are not manufactured with the ability to self-disinfect. Thus, medical imaging equipment has to be wiped with chemical/disinfection agents/reagents. In light of the COVID-19 pandemic, the inventors have recognized the need for systematically and thoroughly disinfecting/decontaminating imaging equipment that are used to aid the diagnosis and interventions of COVID-19 patients.

The equipment disinfections/decontaminators can be done between each patient imaging examination. Known chemical decontamination protocols can add minutes if not hours to the workflow, slowing down and adding additional cost to the patient diagnosis and treatment, not to mention causing suboptimal efficiencies in imaging protocols/procedures. In scenarios such as the COVID-19 pandemic, diagnosis speed and imaging efficiency are very important.

In general, medical imaging equipment, such as an X-ray device, a magnetic resonance imaging (MRI) device, a Computed Tomography (CT) device, and a Positron Emission Tomography (PET) device, and combination devices (e.g., PET/CT devices, MRI/PET devices) are fitted with at least one UV light source that is utilized to disinfect the medical imaging equipment prior to, after, and/or between patient imaging procedures. The medical imaging equipment and the UV light source form a disinfecting medical image system. In one embodiment, the UV light source can be manually or automatically placed (1) into an imaging area where a patient remains during his/her imaging and/or (2) around the imaging area outside of where a patient remains during his/her imaging. In another embodiment, the UV light source is built into the medical imaging equipment to avoid the need to move the UV light source between disinfections.

In an alternate embodiment, the medical imaging equipment discussed above is further configured to include a control system for selectively cleaning areas of the medical imaging equipment more intensely (e.g., for a longer duration or with in a higher-power cleaning mode) as compared with other areas.

Alternate medical imaging equipment also can be used in conjunction with disinfections systems discussed herein. For example, ultrasound imaging devices are applied directly to the outside of a patient, providing for close patient and medical provider interaction. Given that the interior of the ultrasound imaging devices are not exposed to patients, only the exterior of those devices need to be disinfected. In one such embodiment, an ultrasound imaging device is placed into a UV-disinfecting cabinet or storage device which disinfects the ultrasound imaging device while not in use. In an alternate embodiment, the ultrasound imaging device is brought (manually or robotically) into close proximity with a UV light source that disinfects the outside of ultrasound imaging device.

### BRIEF DESCRIPTION OF THE DRAWINGS

FIG. 1A shows a general stand up CT scanner system configuration.

FIG. 1B shows a general stand up CT scanner system configuration with a subject standing in place.

FIG. 1C shows an example of a UV light emitting ring that may be attached to a stand up CT scanner system.

FIG. 1D shows a UV light emitting ring emitting UV as it is coming out of the floor.

FIG. 1E shows a UV light emitting ring emitting UV as it is moving upwards from the bottom of the stand up CT scanner system.

FIG. 1F shows a UV light emitting ring emitting UV at the top of the stand up CT scanner system.

FIG. 1G shows a UV light emitting ring emitting UV at the top of the stand up CT scanner system, and a curtain to block UV from being exposed outside the stand up CT scanner system.

FIG. 2A shows a first view of a general PET scanner system configuration.

FIG. 2B shows a second view of a general PET scanner system configuration.

FIG. 2C shows a UV light emitting apparatus moved onto a bed in a general PET scanner system.

FIG. 2D shows a robotic arm that has placed a UV light emitting apparatus in a general PET scanner system, where UV is being emitted (turned on).

FIG. 2E shows a robotic arm that has placed a UV light emitting apparatus outside of a general PET scanner system, where UV is not being emitted (turned off).

FIG. 2F shows UV light emitting objects that have been periodically placed at junctions of a general PET scanner system.

FIG. 3A shows a general CT scanner system configuration, where the object/patient may lay down.

FIG. 3B shows a UV light emitting object placed in a general CT scanner system.

FIG. 3C shows a UV light emitting object attached to a robotic arm, where the robotic arm has placed the UV light emitting object in the general CT scanner system.

FIG. 3D shows a UV light emitting object attached to a robotic where the robotic arm has placed the UV light emitting object outside the general CT scanner system.

FIG. 4A shows a general MRI system configuration.

FIG. 4B shows a UV light emitting object moved onto the bed of a general MRI system before it enters the general MRI system.

FIG. 4C shows a UV light emitting object moved onto the bed of a general MRI system after it has entered the general MRI system.

FIG. 4D shows a UV light emitting object attached to a robotic arm, where the robotic arm has placed the UV light emitting object at a first position in the general MRI system.

FIG. 4E shows a UV light emitting object attached to a robotic arm, where the robotic arm has placed the UV light emitting object at a second position in the general MRI system.

FIG. 5A shows a general X-ray fluoroscopy configuration.

FIG. 5B shows a general X-ray fluoroscopy with a UV light emitting object moved onto a patient bed.

FIG. 5C shows a general X-ray fluoroscopy with a UV light emitting object placed in the general X-ray fluoroscopy configuration by a robotic arm.

FIG. 6A shows a general ultrasound configuration.

FIG. 6B shows ultrasound equipment placed inside of a cabinet or storage device, the cabinet or storage device having UV light emitting sources attached throughout to emit UV rays onto the ultrasound equipment.

FIG. 6C shows UV light emitting sources attached to robotic arms, the robotic arms being able to move the UV light emitting sources so that the ultrasound equipment can be decontaminated.

FIG. 7A is a diagram showing an outer appearance of a C-arm device according to the embodiment.

FIG. 7B is a diagram showing an example of an operation of the C-arm device according to the embodiment.

FIG. 7C is a diagram showing an example of an operation of the C-arm device according to the embodiment.

FIG. 7D is a diagram showing an example of an operation of the C-arm device according to the embodiment.

FIG. 7E is a diagram showing an outer appearance of an X-ray diagnostic apparatus according to the embodiment.

### DETAILED DESCRIPTION

In light of the above mentioned problems, medical imaging equipment is provided with at least one of an internal and an external UV-based disinfection system. Such a UV-based disinfection system may be implemented with a composite UV viral and bacterial disinfection system with the capability to use ultraviolet (UV) radiation/light with broad spectrum light. The UV light spectrum may include UV-C, UV-B and anti-bacterial UV-A to optimize their germ killing efficiency. UV-C light targets the DNA and RNA of microbes and destroys the ability of microorganisms to reproduce/replicate. The high energy short wavelength UV-C light is absorbed in cellular RNA and DNA, leading to damaged nucleic acids. This prevents microorganisms from being infective and impair their ability to reproduce/replicate. The absorption of UV-C energy forms new chemical bonds between nucleotides, creating double bonds or “dimers”. Dimerization of molecules, particularly thymine, is the most common type of damage inflicted by UV-C light in microorganisms. Formation of thymine dimers in the DNA of bacteria and viruses prevents replication and their ability to infect. Thus, in one embodiment, the UV-based disinfection system uses UV-C (254 nm) for disinfection.

The techniques and structures described herein can be applied to a variety of medical imaging equipment. That medical imaging equipment includes, but is not limited to, computed tomography (CT), positron emission tomography (PET), magnetic resonance imaging (MRI), X-ray imaging technologies including angiography, c-arm and mammography, ultrasound, and dual energy X-ray absorptiometry (DEXA) imaging. Various embodiments will be described to illustrate some of the above mentioned techniques and structures. To prevent exposure to humans, a motion sensor interrupts operation if motion is detected during operation. Furthermore, as UV-C does not penetrate glass, in at least one embodiment operator room windows are not curtained.

In one embodiment, the above-mentioned techniques and concepts are applied to a stand up CT scanner system. According to one embodiment, a stand up X-ray computed tomography apparatus includes a gantry body 11, a column 13, and a beam 14. The gantry body includes a bore to include a field of view, and also includes an X-ray tube and an X-ray detector. The column supports the gantry body so that the gantry body is vertically movable with a central axis of the bore extending vertically to a floor face. The fixing equipment fixes a subject holder so that the subject holder is located on a passage of the bore and partially in the bore in a phase of attaching a subject to the subject holder.

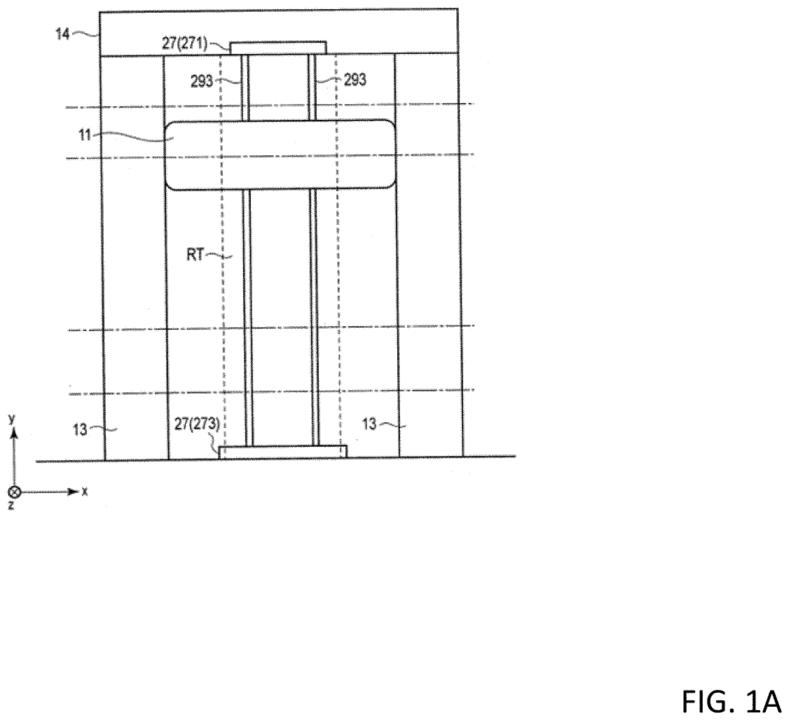

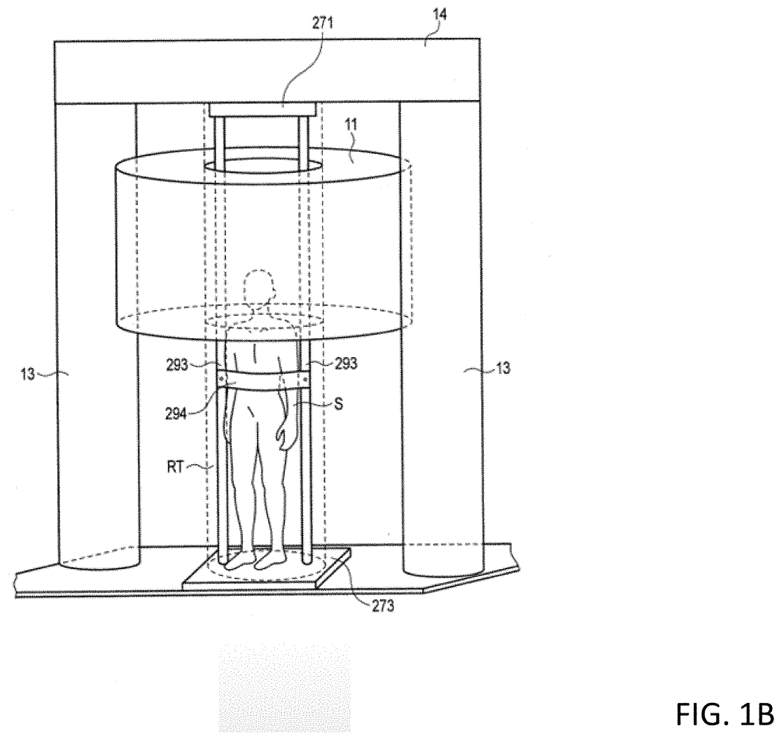

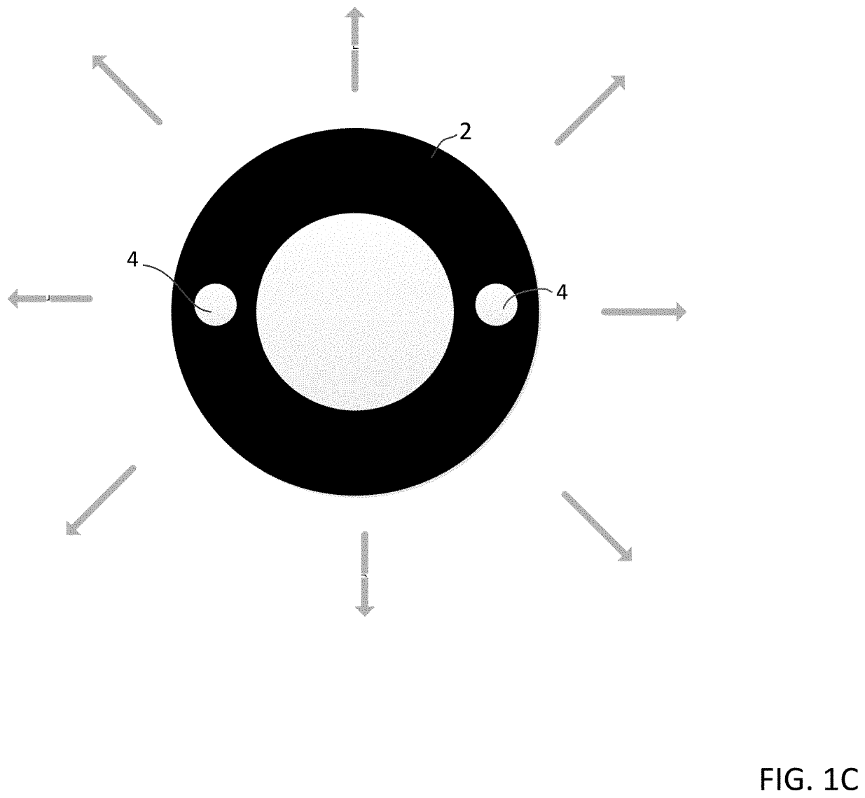

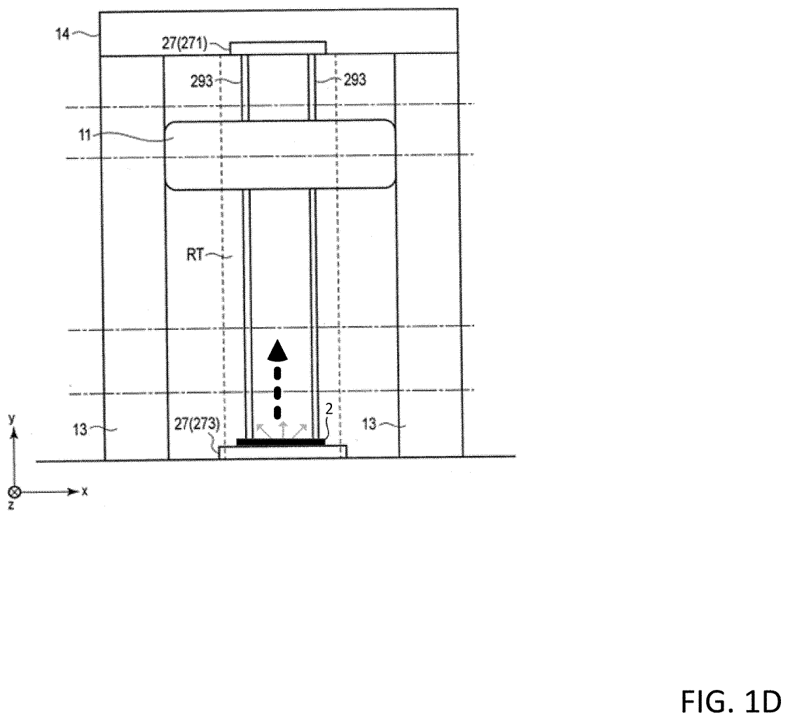

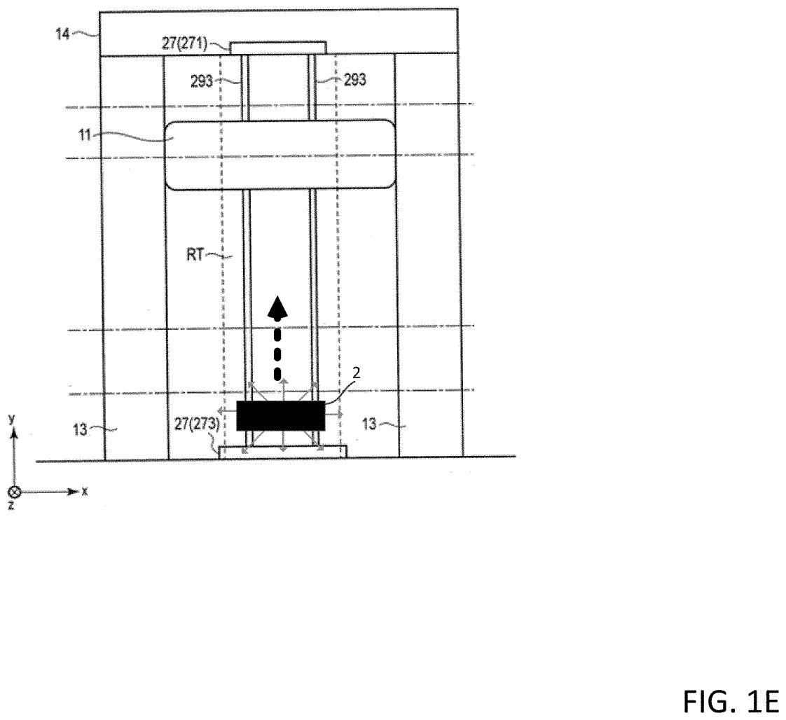

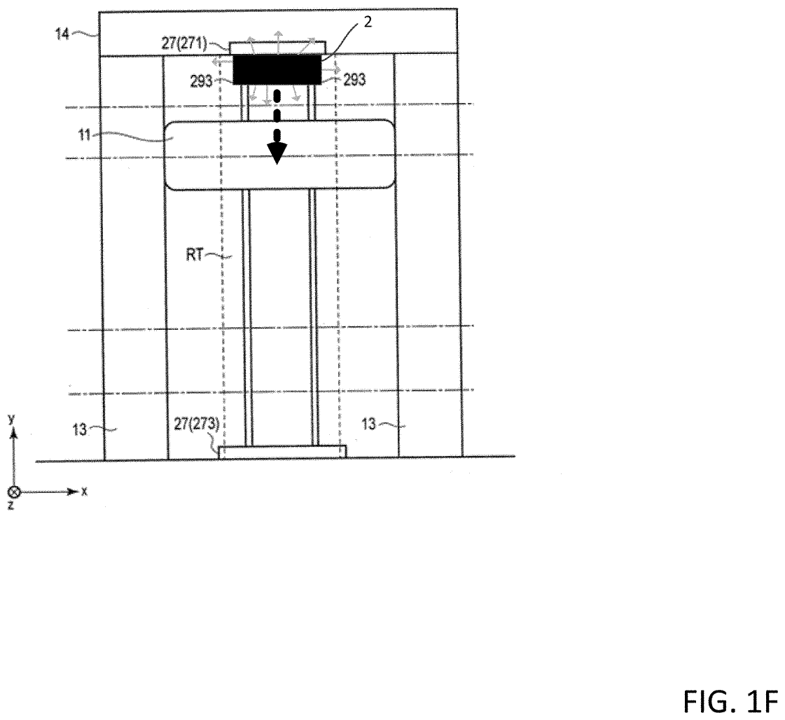

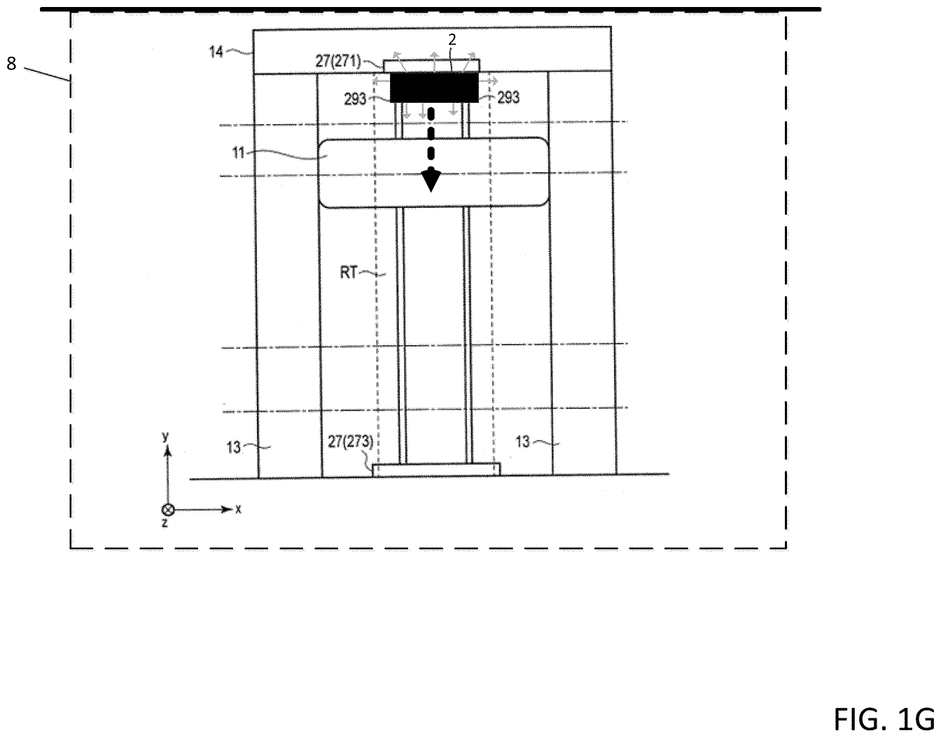

FIG. 1A shows one example of a standup CT scanner with gantry 10 that includes a gantry body 11, a column 13, and a beam 14. In addition, the CT scanner includes a subject holder including two pillars 293 that are interior to a moving gantry 11, as disclosed in U.S. patent application Ser. No. 15/711,650. FIG. 1B shows the dual pillar subject holder 293 of FIG. 1A together with a subject S. As shown in FIGS. 1A and 1B, the two pillars 293 are fixed by the holder fixing equipment 27. Specifically, the upper ends of the two pillars 293 are fixed by the upper fixing part 271, and the lower ends of the two pillars 293 are fixed by the lower fixing part 273. The two pillars 293 may be provided in any arrangement within the passage RT. For example, the upper fixing part 271 and the lower fixing part 273 fix the two pillars 293 so that the two pillars 293 are located on the respective sides of the subject S as shown in FIGS. 1A and 1B. In order to more reliably secure the subject S to the two pillars 293, an accessory fastener 294, such as a band, may be detachably provided to the two pillars 293 as shown in FIG. 1B. With the accessory fastener 294, the subject S can be fastened to the two pillars 293.

A UV light emitting apparatus may be incorporated into or attached to the stand up CT system to emit UV and disinfect the system. FIG. 1C shows an example of a UV light emitting apparatus 2 that may be attached to the CT system using the existing pillars 293. The UV light emitting apparatus can have holes 4 for the two pillars 293 to pass through so that the UV light emitting apparatus can move vertically about the pillars as it emits UV rays (i.e., disinfects).

In FIG. 1D, the UV light emitting apparatus 2 is coming from out of the floor, and it is partially out of the floor and heading in an upwards direction. As the UV light emitting apparatus 2 moves upwards, UV rays are emitted inward toward the center of the emitting apparatus 2 (e.g., to disinfect the pillars 293) and outward from the emitting apparatus 2 to disinfect the inside of the gantry 10 including the gantry body 11, the column 13, and the beam 14.

In FIG. 1E the UV light emitting apparatus 2 has fully come out of the floor, and is heading upwards from the lower end to the upper end of the two pillars 293. As the UV light emitting apparatus 2 moves upwards, UV rays may emit out in all directions to disinfect. In FIG. 1F, the UV light emitting apparatus 2 has reached the top of the two pillars 293, and is heading downwards from the upper end to the lower end. As the UV light emitting apparatus 2 moves downwards, UV rays may continue to emit out in all directions.

In FIG. 1G, a UV impenetrable screen/curtain 8 has been incorporated into the device. This can help prevent harmful UV exposure to, for instance, nearby patients and medical professionals.

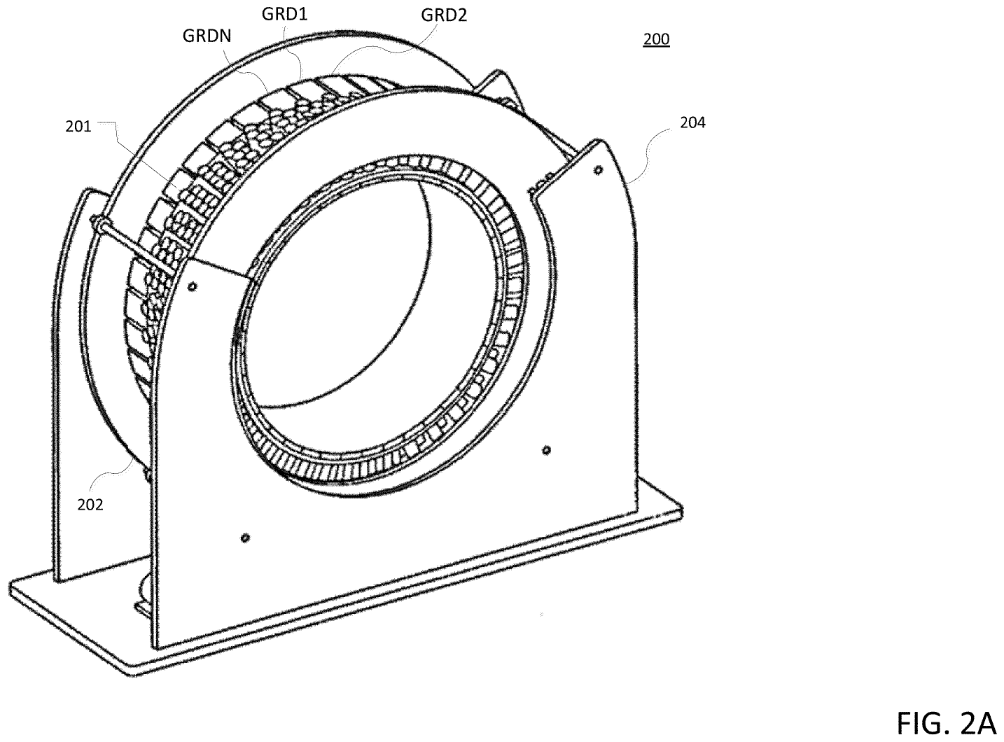

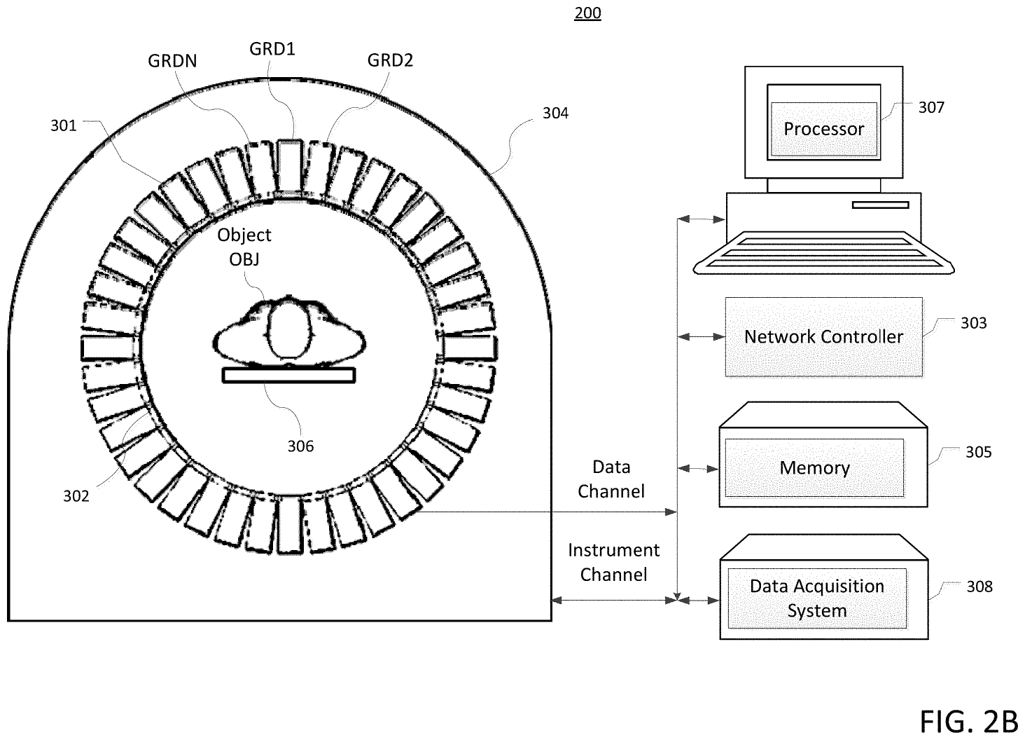

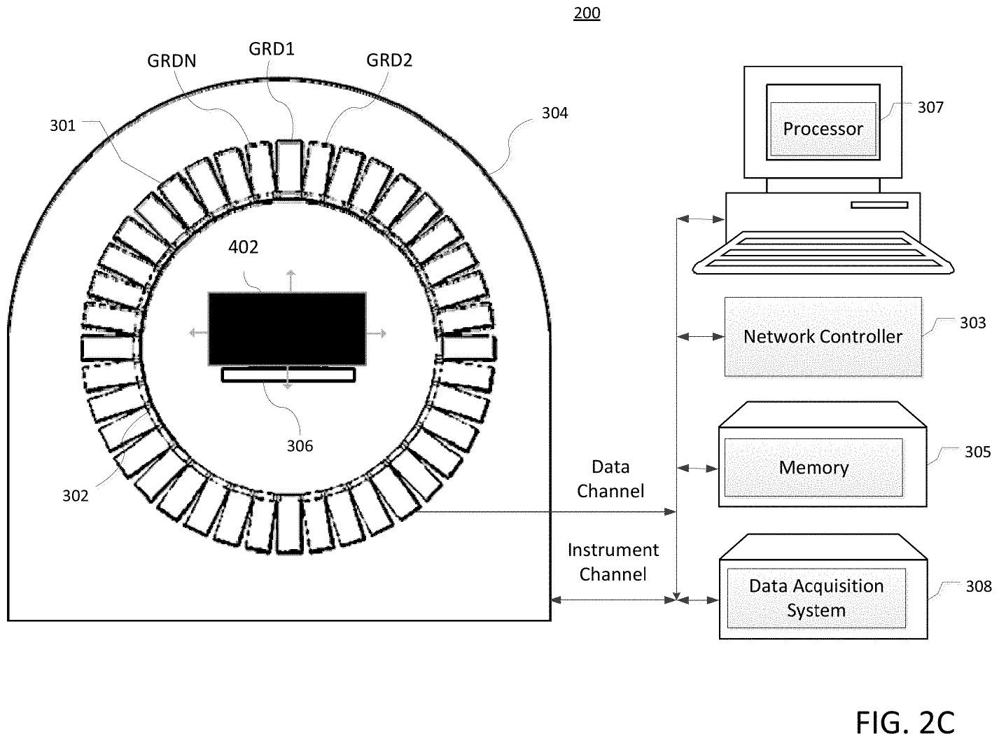

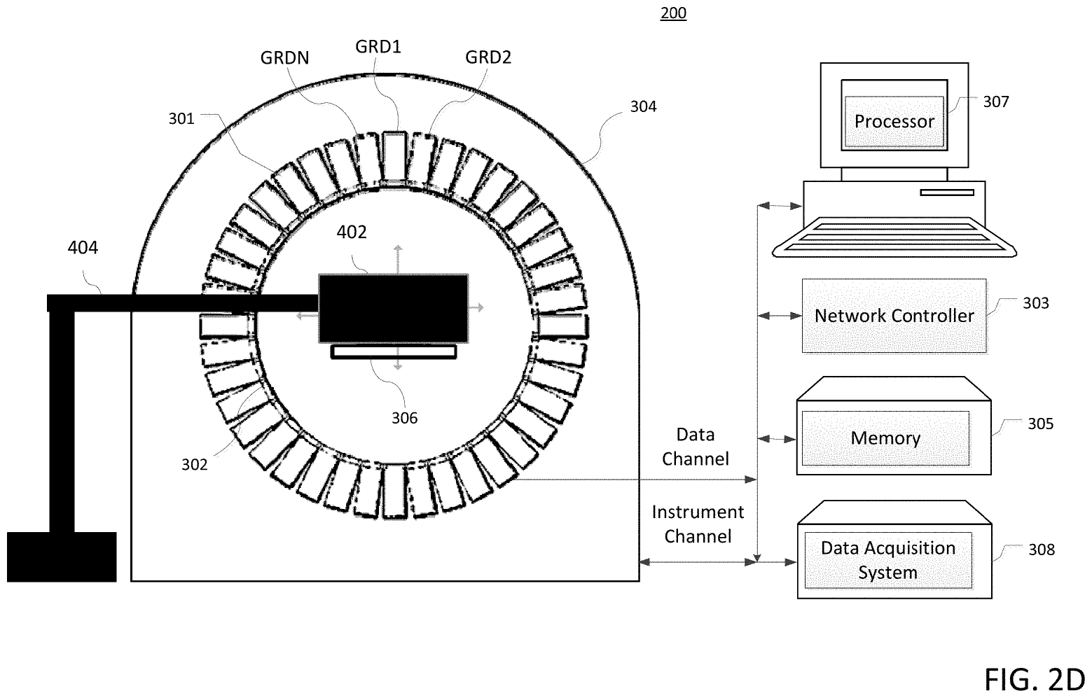

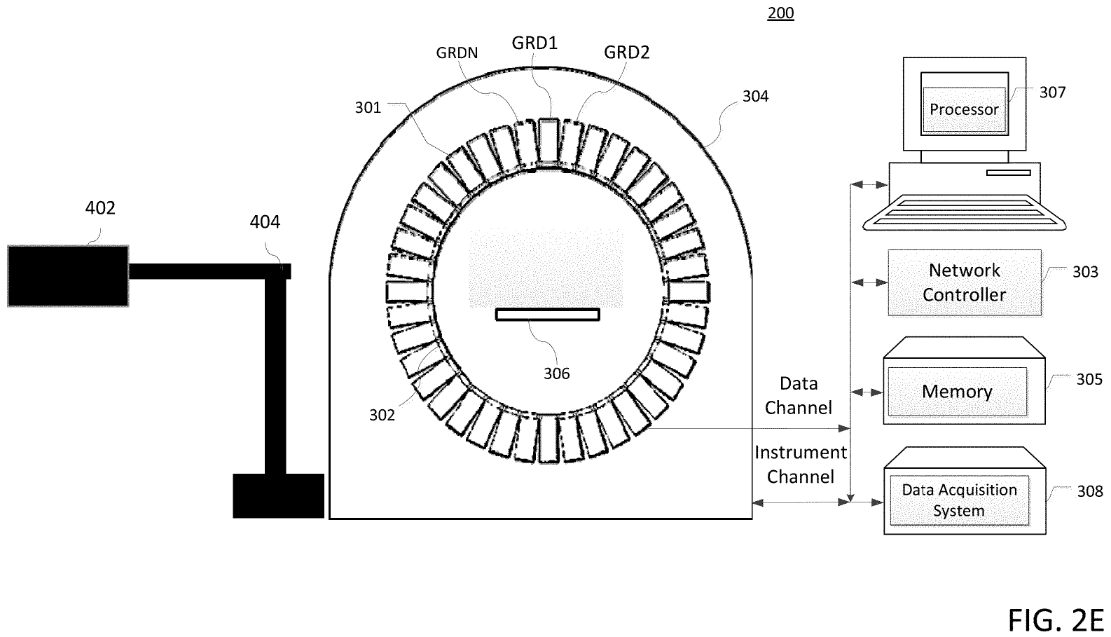

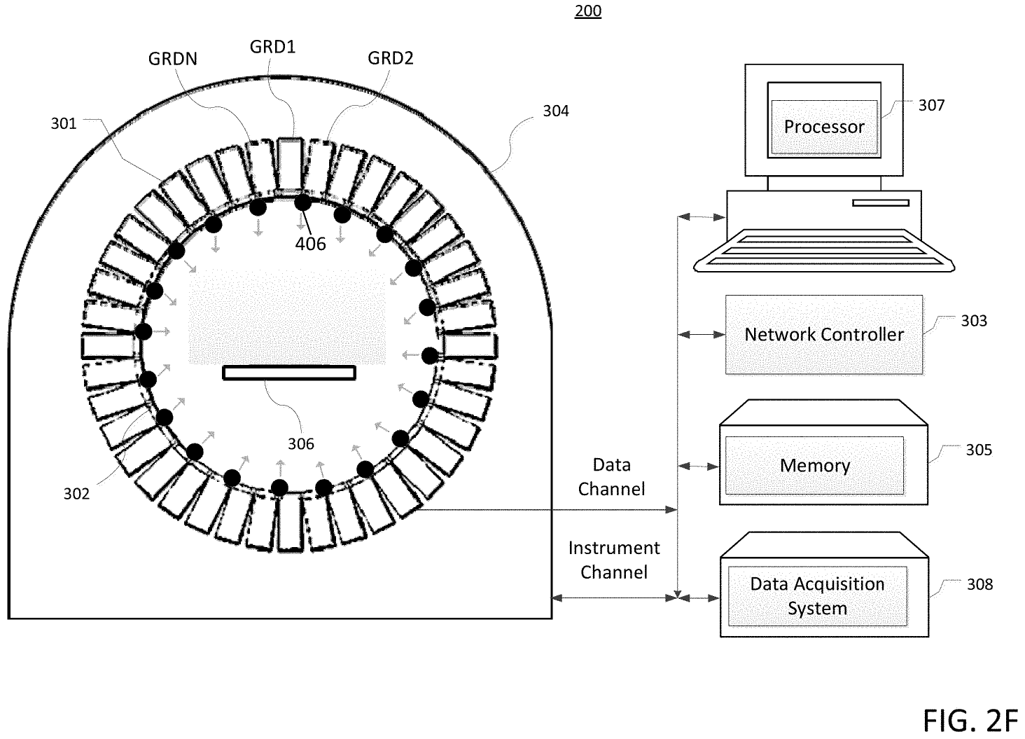

In one embodiment, it can be appreciated that the methods of the present disclosure may be implemented within a PET scanner. FIG. 2A and FIG. 2B show a PET scanner 200 including a number of gamma-ray detectors (GRDs) 201 (e.g., GRD₁, GRD₂, through GRD_{N}) that are each configured as rectangular detector modules. According to one implementation, each PET detector ring, which forms a circular bore 202 about a gantry 204 including a number of GRDs (e.g., 40 or 48). The translation of each PET detector ring may be accomplished by manual manipulation and/or motorized manipulation. The GRDs include scintillator crystal arrays for converting the gamma rays into scintillation photons (e.g., at optical, infrared, and ultraviolet wavelengths), which are detected by photodetectors.

FIG. 2B shows a schematic view of a PET scanner system having GRDs arranged to detect gamma-rays emitted from an object OBJ (e.g., a patient). The GRDs can measure the timing, position, and energy corresponding to each gamma-ray detection. In one implementation, the gamma-ray detectors are arranged in a PET detector ring, as shown in FIG. 2A and FIG. 2B. It can be appreciated that the single PET detector ring of FIG. 2B can be extrapolated to include any number of PET detector rings along an axial length of the PET scanner. The detector crystals can be scintillator crystals, which have individual scintillator elements arranged in a two-dimensional array and the scintillator elements can be any known scintillating material. The PMTs can be arranged such that light from each scintillator element is detected by multiple PMTs to enable Anger arithmetic and crystal decoding of scintillation event.

FIG. 2B shows an example of the arrangement of the PET scanner 200, in which an object (e.g., a possibly infected person) rests on a table 306 and the GRD modules GRD₁ through GRD_{N} are arranged circumferentially around the object OBJ (e.g., a patient) and the table 306. The GRDs may comprise a PET detector ring and may fixedly-connected to a circular bore 302 that is fixedly-connected to a gantry 304. The gantry 304 houses many parts of the PET scanner. The gantry 304 of the PET scanner also includes an open aperture, defined by the cylindrical bore 302, through which the object OBJ (e.g., a patient) and the table 306 can pass, and gamma-rays emitted in opposite directions from the object OBJ (e.g., a patient) due to an annihilation event can be detected by the GRDs and timing and energy information can be used to determine coincidences for gamma-ray pairs.

In FIG. 2B, circuitry and hardware is also shown for acquiring, storing, processing, and distributing gamma-ray detection data. The circuitry and hardware include a processor 307, a network controller 303, a memory 305, and a data acquisition system (DAS) 308. The PET imager also includes a data channel that routes detection measurement results from the GRDs to the DAS 308, the processor 307, the memory 305, and the network controller 303. The data acquisition system 308 can control the acquisition, digitization, and routing of the detection data from the detectors. In one implementation, the DAS 308 controls the movement or the table 306. The processor 307 performs functions including pre-reconstruction processing of the detection data, image reconstruction, and post-reconstruction processing of the image data.

In one embodiment, a UV light emitting object/apparatus is placed in the PET scanner system to emit UV rays and disinfect the system. For example, as shown in FIG. 2C, a UV light emitting box 402 may be moved onto the table 306; the box 402 may emit UV in all directions when turned on. Further, the box 402 can be moved onto top of the table 306, and the table 306 could move to ensure that the entire PET scanner system has had UV exposure (i.e. been cleaned).

FIG. 2D and FIG. 2E illustrate another embodiment, where a robotic arm 404 is used to move a LV light emitting box 402 in and out of the PET scanner system. In FIG. 2D, the robotic arm 404 has moved the UV light emitting box 402 near the table 306; in this instance, UV rays may be emitting (and thus disinfecting). In FIG. 2E, the robotic arm 404 has moved such that the UV light emitting box 402 is away from the table 306 now that disinfecting has been completed; in this case, the UV rays may be turned off and not emitting.

FIG. 2F illustrates another embodiment. In this example, UV light emitting sources 406 are periodically placed at junctions on the PET scanner, each UV light emitting source may emits UV rays to the various surfaces of the PET scanner systems when turned on. In FIG. 2F, there are 20 UV light emitting sources 406, but in other embodiments more or less can be used.

In addition, for additional safety, a curtain (not shown but similar to the curtain of FIG. 1G) is added to any of the configurations shown in FIG. 2C-FIG. 2F to protect against unnecessary and/or unwanted UV radiation.

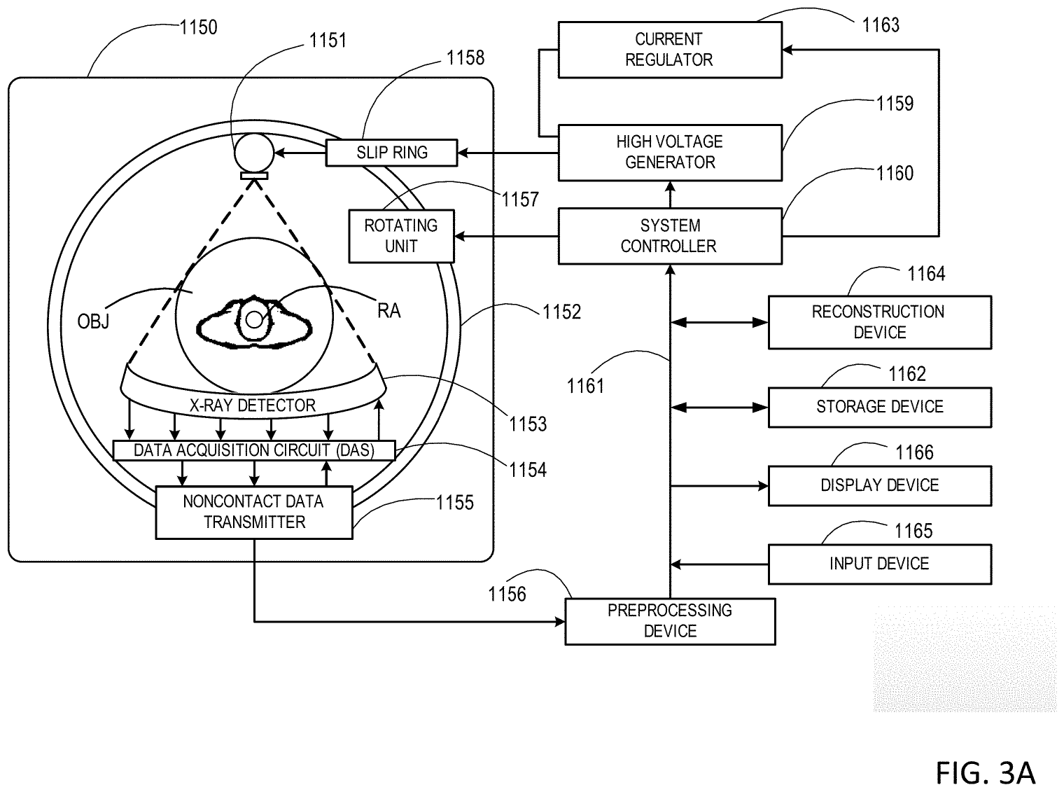

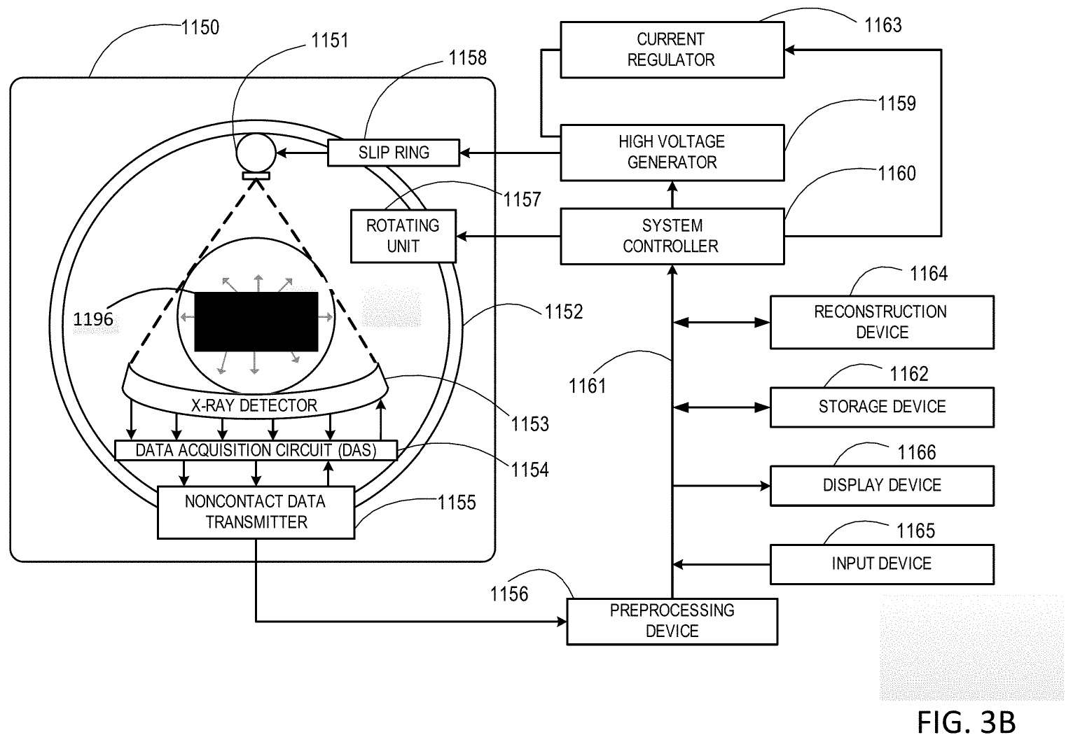

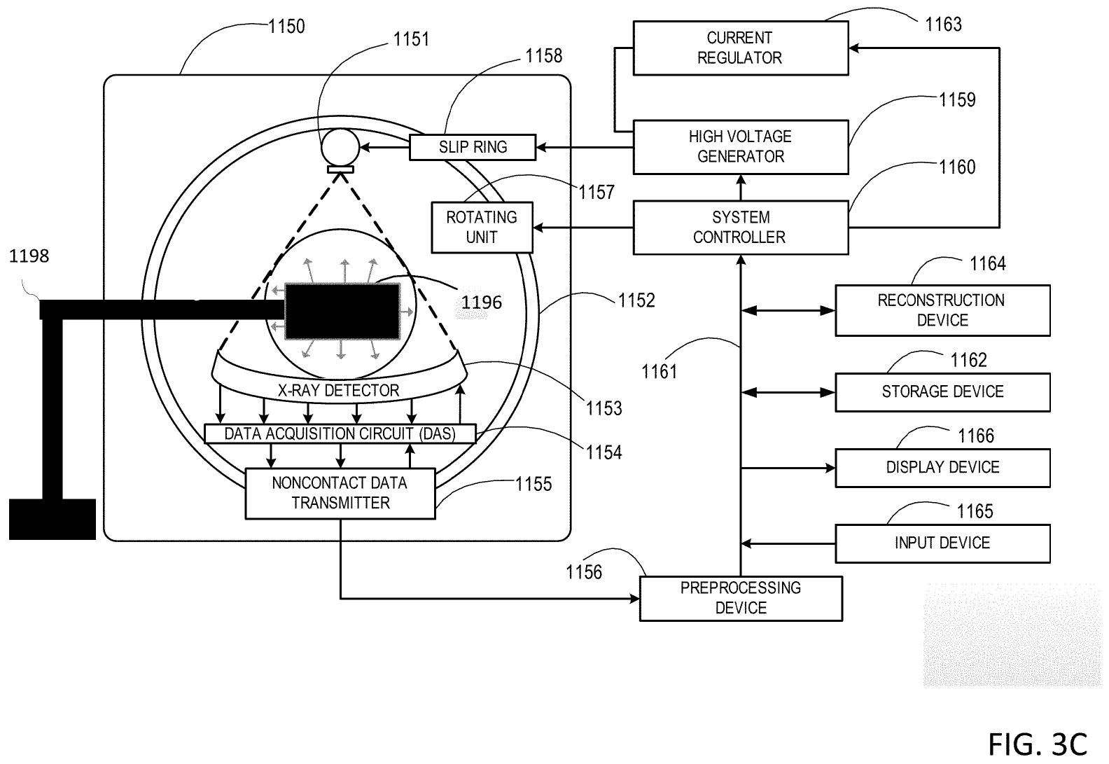

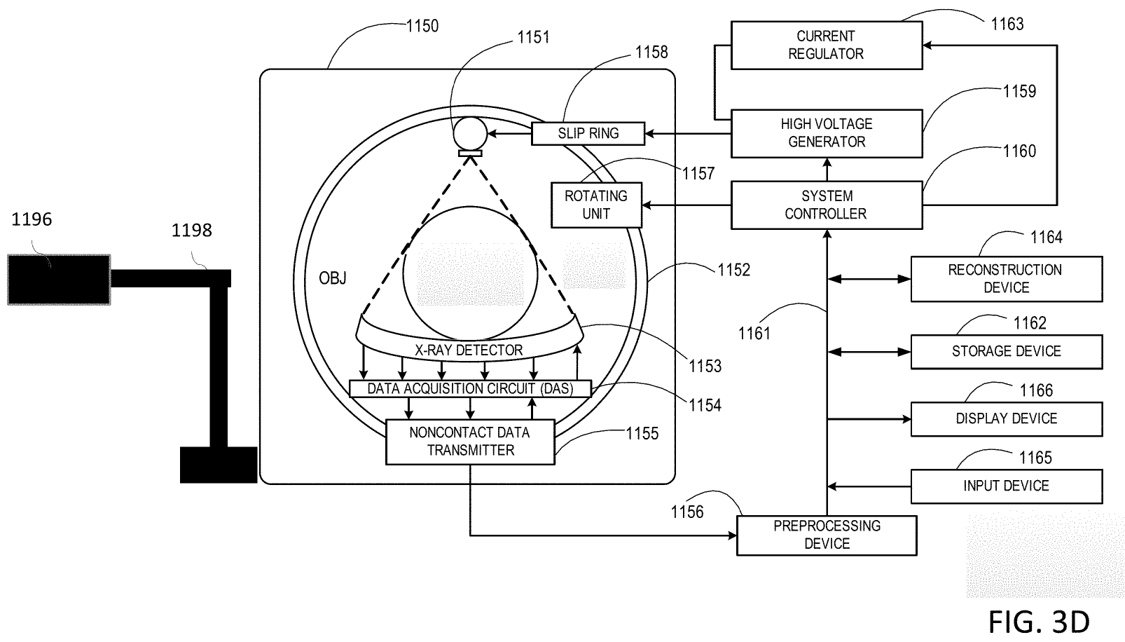

According to an alternate embodiment, the disinfection techniques and structures can be applied to a CT apparatus or scanner. FIG. 3A illustrates an implementation of a horizontal radiography gantry included in a CT apparatus or scanner. This CT scanner differs from the CT scanner shown in FIG. 1A and FIG. 1B in that it is not a stand up CT scanner system. As shown in FIG. 3A, a radiography gantry 1150 (illustrated from a side view) includes an X-ray tube 1151, an annular frame 1152, and a multi-row or two-dimensional-array-type X-ray detector 1153. The X-ray tube 1151 and X-ray detector 1153 are diametrically mounted across an object OBJ (e.g., a patient) on the annular frame 1152, which is rotatably supported around a rotation axis RA. A rotating unit 1157 rotates the annular frame 1152 at a high speed, such as 0.4 sec/rotation, while the object OBJ (e.g., a patient) is being moved along the axis RA into or out of the illustrated page.

An embodiment of an X-ray CT apparatus according to the present inventions will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present inventions can be applied to either type. In this case, the rotate/rotate-type, which is currently the mainstream, will be exemplified.

The multi-slice X-ray CT apparatus further includes a high voltage generator 1159 that generates a tube voltage applied to the X-ray tube 1151 through a slip ring 1158 so that the X-ray tube 1151 generates X-rays. An X-ray detector 1153 is located at an opposite side from the X-ray tube 1151 across the object OBJ (e.g., a patient) for detecting the emitted X-rays that have transmitted through the object OBJ (e.g., a patient). The X-ray detector 1153 further includes individual detector elements or units and may be a photon-counting detector. In the fourth-generation geometry system, the X-ray detector 1153 may be one of a plurality of detectors arranged around the object OBJ (e.g., a patient) in a 360° arrangement.

The CT apparatus further includes other devices for processing the detected signals from the X-ray detector 1153. A data acquisition circuit or a Data Acquisition System (DAS) 1154 converts a signal output from the X-ray detector 1153 for each channel into a voltage signal, amplifies the signal, and further converts the signal into a digital signal. The X-ray detector 1153 and the DAS 1154 are configured to handle a predetermined total number of projections per rotation (TPPR).

The above-described data is sent to a preprocessing device 1156, which is housed in a console outside the radiography gantry 1150 through a non-contact data transmitter 1155. The preprocessing device 1156 performs certain corrections, such as sensitivity correction, on the raw data. A memory 1162 stores the resultant data, which is also called projection data at a stage immediately before reconstruction processing. The memory 1162 is connected to a system controller 1160 through a data/control bus 1161, together with a reconstruction device 1164, input device 1165, and display 1166. The system controller 1160 controls a current regulator 1163 that limits the current to a level sufficient for driving the CT system. In an embodiment, the system controller 1160 implements optimized scan acquisition parameters, as described above.

As shown in FIG. 3B, a UV light emitting apparatus 1196 can be automatically or manually placed in the CT scanner system. The UV light emitting apparatus then can emit UV rays to disinfect the CT system.

Further, the UV light emitting apparatus can be moved in and out of the CT scanner system with a robotic arm, as illustrated in FIG. 3C and FIG. 3D. In FIG. 3C, the UV light emitting apparatus 1196 has been moved into or near the CT scanner system with a robotic arm 1198, and the UV light emitting apparatus 1196 may be emitting UV rays. In FIG. 3D, the UV light emitting apparatus 1196 has been moved to outside of the CT scanner system by the robotic arm 1198, and the UV light emitting apparatus 1196 may not be emitting UV rays.

In another embodiment, a UV impenetrable screen/curtain may be incorporated into the device. This can help prevent harmful UV exposure to, for instance, nearby patients and medical professionals.

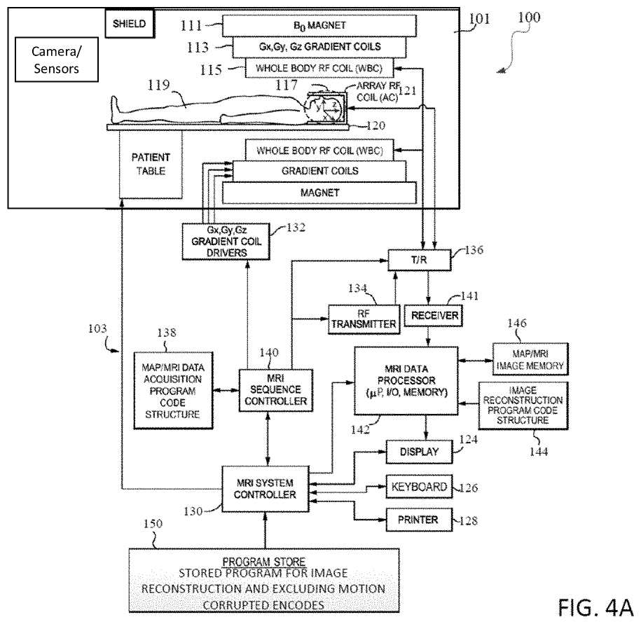

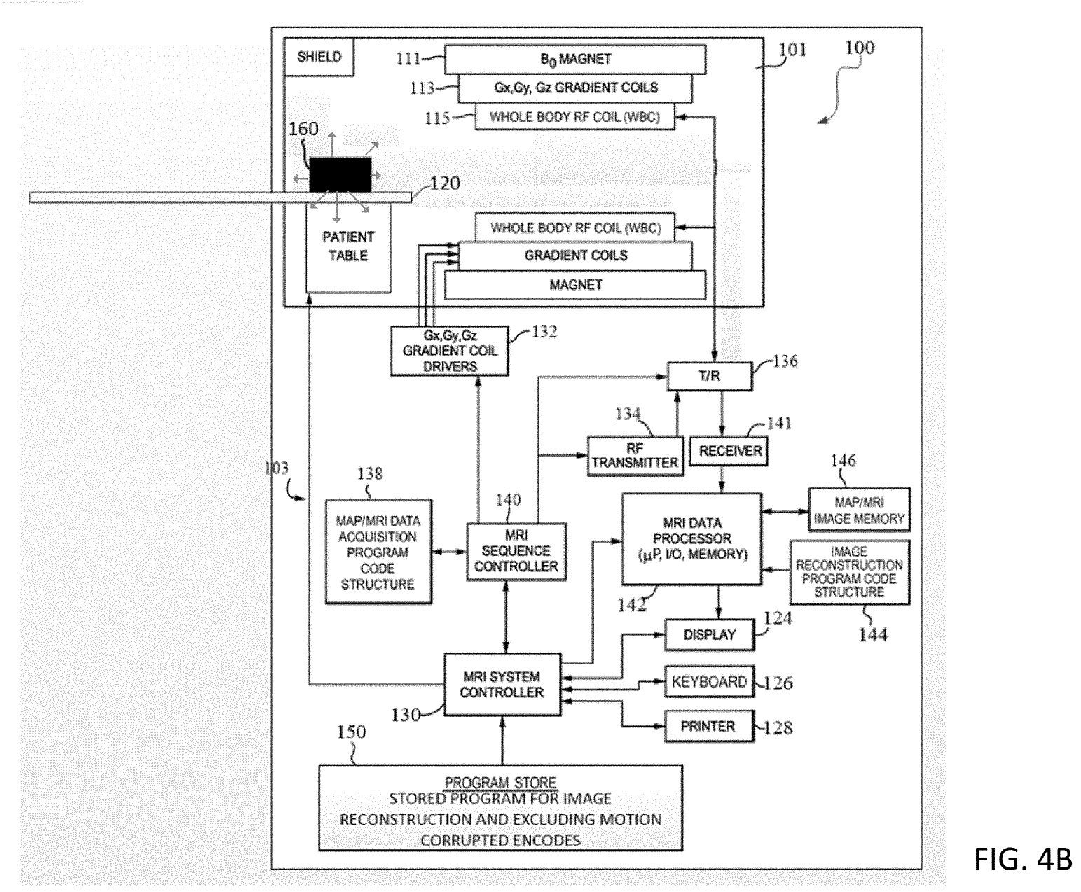

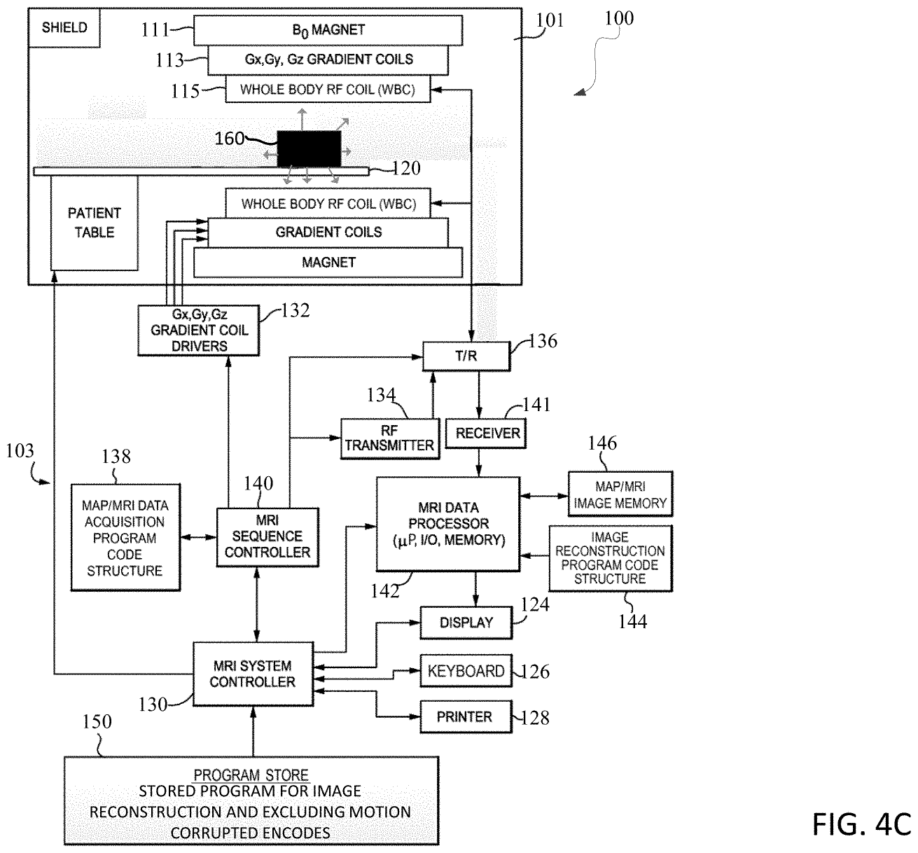

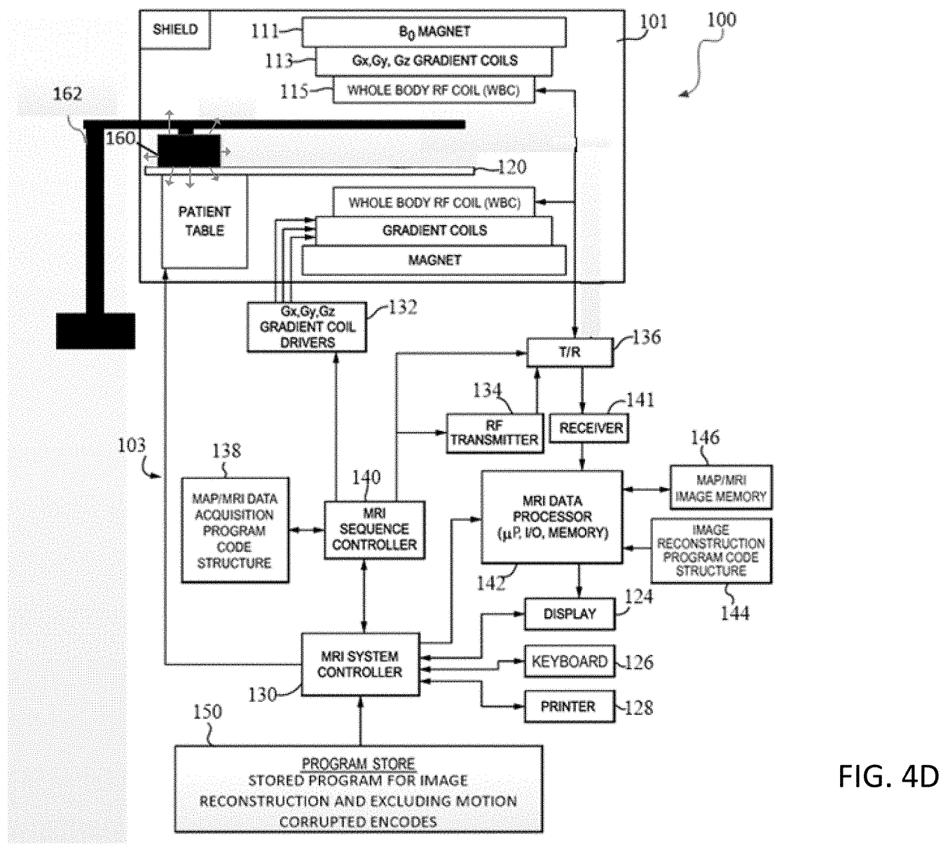

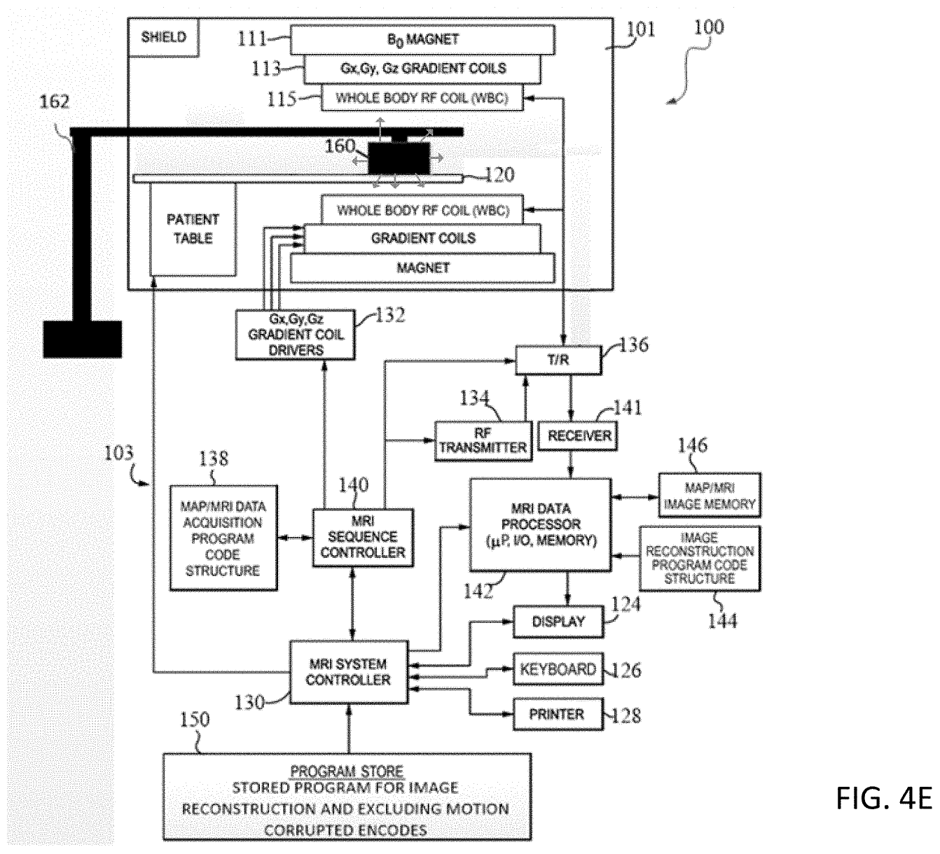

The above-mentioned techniques and structures can be applied in an MRI. FIG. 4A shows a non-limiting example of a magnetic resonance imaging (MRI) system 100. The MRI system 100 depicted in FIG. 4A includes a gantry 101 (shown in a schematic cross-section) and various related system components 103 interfaced therewith. At least the gantry 101 is typically located in a shielded room. The MRI system geometry depicted in FIG. 4A includes a substantially coaxial cylindrical arrangement of the static field B0 magnet 111, a Gx, Gy, and Gz gradient coil set 113, and a large whole-body RF coil (WBC) assembly 115. Along a horizontal axis of this cylindrical array of elements is an imaging volume 117 shown as substantially encompassing the head of a patient 119 supported by a patient table 120.

One or more smaller array RF coils 121 can be more closely coupled to the patient's head (referred to herein, for example, as “scanned object” or “object”) in imaging volume 117. As those in the art will appreciate, compared to the WBC (whole-body coil), relatively small coils and/or arrays, such as surface coils or the like, are often customized for particular body parts (e.g., arms, shoulders, elbows, wrists, knees, legs, chest, spine, etc.). Such smaller RF coils are referred to herein as array coils (AC) or phased-array coils (PAC). These can include at least one coil configured to transmit RF signals into the imaging volume, and a plurality of receiver coils configured to receive RF signals from an object, such as the patient's head, in the imaging volume. Alternatively, whole-body coils systems can be used.

The MRI system 100 includes a MRI system controller 130 that has input/output ports connected to a display 124, a keyboard 126, and a printer 128. As will be appreciated, the display 124 can be of the touch-screen variety so that it provides control inputs as well. A mouse or other I/O device(s) can also be provided.

The MRI system controller 130 interfaces with a MRI sequence controller 140, which, in turn, controls the Gx, Gy, and Gz gradient coil drivers 132, as well as the RF transmitter 134, and the transmit/receive switch 136 (if the same RF coil is used for both transmission and reception). The MRI sequence controller 140 includes suitable program code structure 138 for implementing MRI imaging (also known as nuclear magnetic resonance, or NMR, imaging) techniques including parallel imaging. MRI sequence controller 140 can be configured for MR imaging with or without parallel imaging. Moreover, the MRI sequence controller 140 can facilitate one or more preparation scan (pre-scan) sequences, and a scan sequence to obtain a main scan magnetic resonance (MR) image (referred to as a diagnostic image). MR data from pre-scans can be used, for example, to determine sensitivity maps for RF coils 115 and/or 121 (sometimes referred to as coil sensitivity maps or spatial sensitivity maps), and to determine unfolding maps for parallel imaging.

The MRI system components 103 include an RF receiver 141 providing input to data processor 142 so as to create processed image data, which is sent to display 124. The MRI data processor 142 is also configured to access previously generated MR data, images, and/or maps, such as, for example, coil sensitivity maps, parallel image unfolding maps, distortion maps and/or system configuration parameters 146, and MRI image reconstruction program code structures 144 and 150.

In one embodiment, as shown in FIG. 4B and FIG. 4C, a UV light emitting apparatus 160 can be moved onto the patient table 120 after the patient table 120 has been at least partially slid out, as shown in FIG. 4B. Then, the patient table 120, now with the UV light emitting apparatus 160, can slide into the MRI system, where the UV light emitting apparatus 160 may be emitting UV rays, as illustrated in FIG. 4C.

In another embodiment, rather than manually placing a UV light emitting apparatus 160 onto the patient table 120 (e.g. by a technician), a robot arm can be used to move the UV light emitting apparatus 160 into and out of the MRI system, as illustrated by FIG. 4D and FIG. 4E. In FIG. 4D, the UV light emitting apparatus 160 is affixed onto a robotic arm 162 and turned on (i.e. emitting UV rays). The robotic arm 162 may then guide the UV light emitting apparatus 160 within the MRI system, as shown in FIG. 4E, so that the various components of the MRI system may get UV exposure, and thus disinfected. In another embodiment, a UV impenetrable screen/curtain may be incorporated into the device.

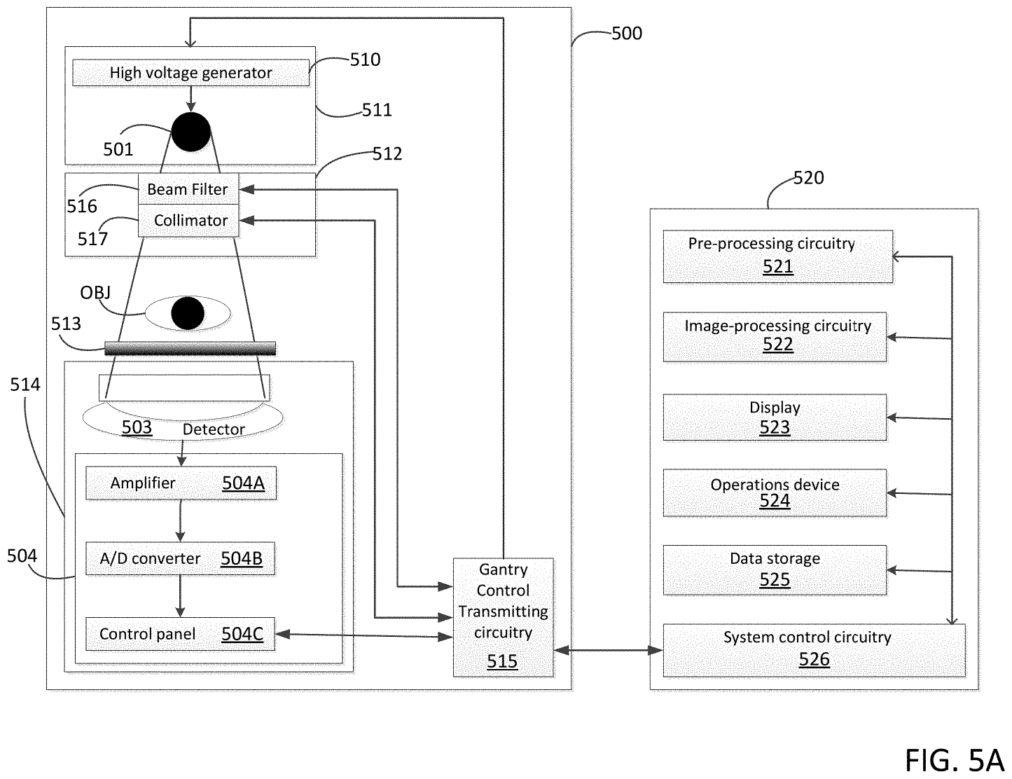

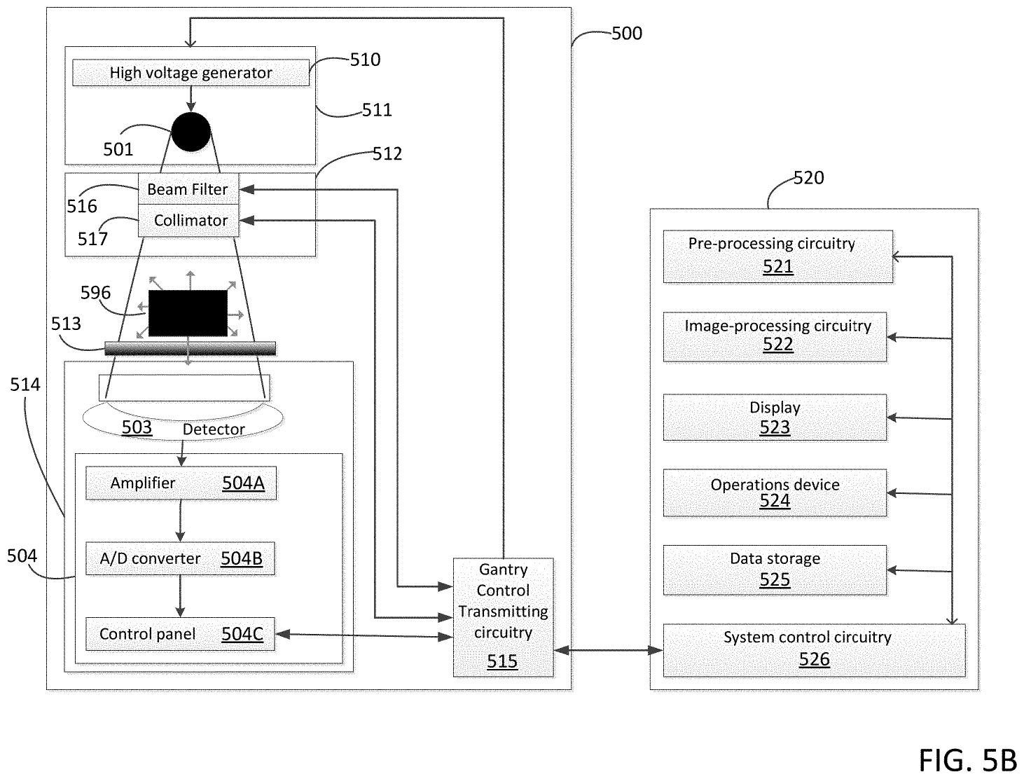

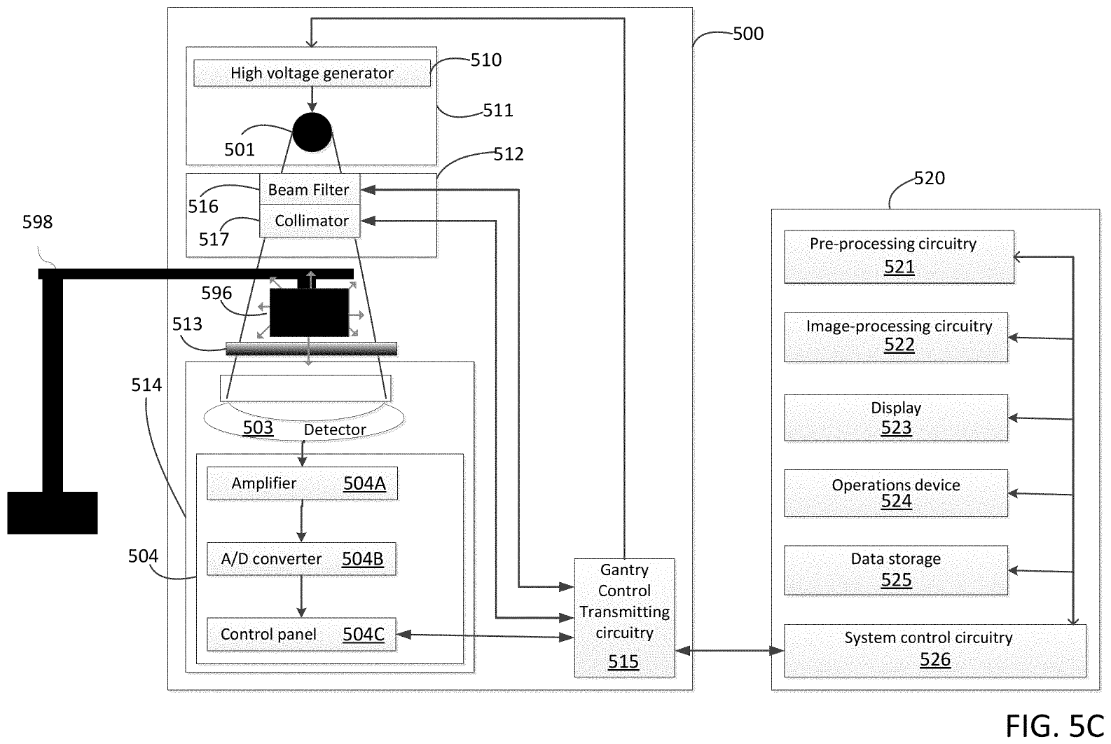

FIG. 5A is an exemplary X-ray apparatus that can be used with the disclosed techniques and structures. The X-ray apparatus includes a gantry 500 and a console 520. The gantry 500 includes an X-ray source system 511, a beam-shaping system 512, a patient table 513, a detection system 514, and a gantry control transmission circuitry 515. The X-ray source system 511 includes a high voltage generator 510 and an X-ray tube 501. The high voltage generator 510 applies a high voltage to the X-ray tube 501 under the control of the gantry control transmission circuitry 515, and supplies a filament current to the X-ray tube 501 under the control of the gantry control transmission circuitry 515. The X-ray tube 501 generates X-rays to irradiate an object OBJ (e.g., a patient) upon receiving a trigger from the high voltage generator 510. The collimation system 512 includes a beam filter/attenuator 516 which modifies the spectrum of the X-ray beam from the X-ray tube 501. A collimator 517 opens and closes in accordance with a field of view selected at the time of the operation. The collimation system 512 forms an X-ray beam and irradiates the object OBJ (e.g., a patient) with X-rays.

The detection system 514 includes a two-dimensional array of detection elements (pixels) configured to absorb the X-ray transmitted through the object OBJ (e.g., a patient) and generate an electrical charge signal proportional to the absorbed X-ray intensity. The electrical signal of each pixel is amplified and converted to a digital number by A/D converters. For example, the detection system 514 includes the detector 503 and a data acquisition system (DAS) 504. The detector 503 detects the X-rays generated from the X-ray tube 501. The detector 503 is equipped with a plurality of detection elements arrayed two-dimensionally. Each detection element detects the X-rays generated from the X-ray tube 501 and generates an electrical signal (current signal) corresponding to the intensity of the detected X-rays.

The generated electrical signal is supplied to the DAS 504. The DAS 504 includes art amplifier 504A, an A/D converter 504B, and a control panel 504C. The DAS 504 reads out electrical signals via the detector 503 and obtains the readout electrical signals, via the control panel 504C. The gantry control transmission circuitry 515 controls the high voltage generator 510, the attenuator 516, the collimator 517, and the control panel 504 to execute X-ray imaging.

The console 520 includes pre-processing circuitry 521, image-processing circuitry 522, a display 523, an operation device 524, data storage 525, and system control circuitry 526. The pre-processing circuitry 521 executes pre-processing, such as sensitivity correction for raw data supplied from the detection system 514, is the gantry control transmission circuitry 515.

The image-processing circuitry 522 can perform the image-processing methods. The display 523 displays the image generated by the image processing circuitry 522. The operation circuitry 524 accepts various types of commands and information inputs from a user, via an input device. The data storage (memory) 525 stores the raw data and various types of data, such as projection data and images. In addition, the data storage 525 stores control programs for the X-ray apparatus, and control programs for performing the image-processing methods described herein. The system control circuitry 526 functions as the main circuitry of the X-ray apparatus. The system control circuitry 526 reads out control programs stored in the data storage 525 and loads the programs into the memory. The system control circuitry 526 controls the respective circuitry in the X-ray apparatus in accordance with the loaded control programs.

In one embodiment, referring to FIG. 5B, a UV light emitting apparatus 596 can be moved onto the patient table 513 and emit UV rays to disinfect the X-ray system. In another embodiment, referring to FIG. 5C, a robotic arm 598 can be used to place the UV light emitting apparatus 596 in various locations throughout the X-ray system such that the system is sufficiently disinfected through UV ray exposure.

In another embodiment, a UV impenetrable screen/curtain may be incorporated into the device. This can help prevent harmful UV exposure to, for instance, nearby patients and medical professionals.

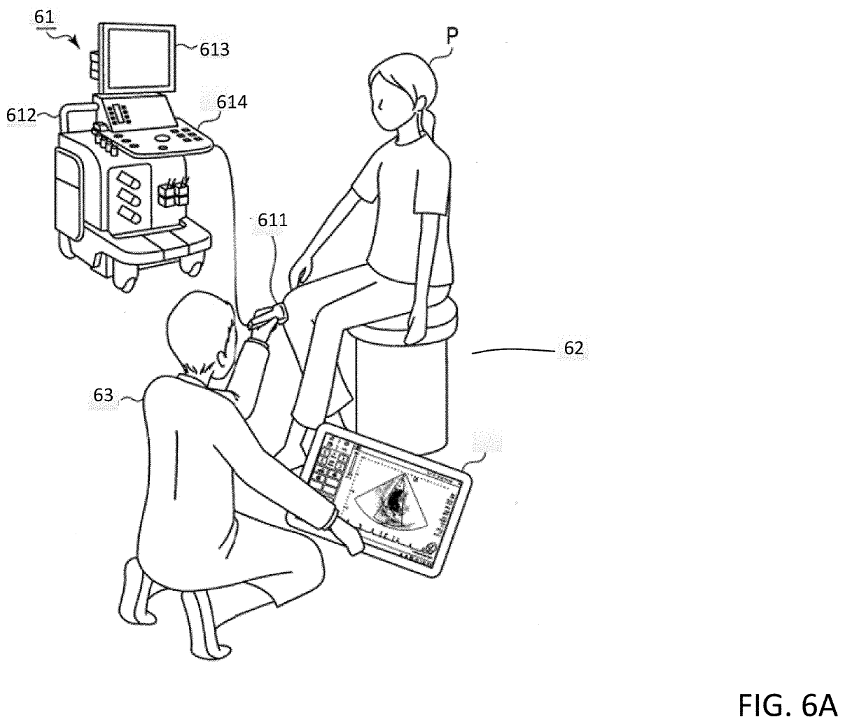

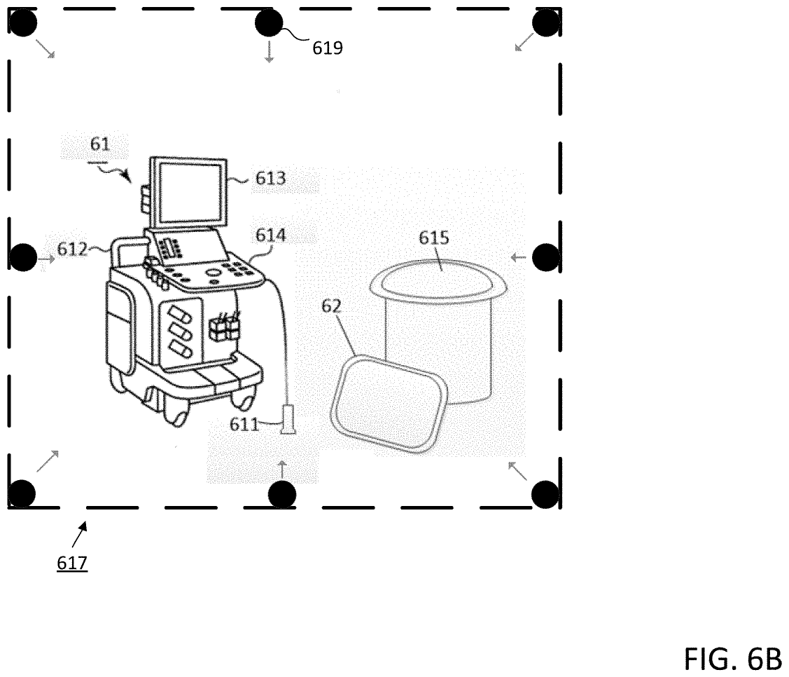

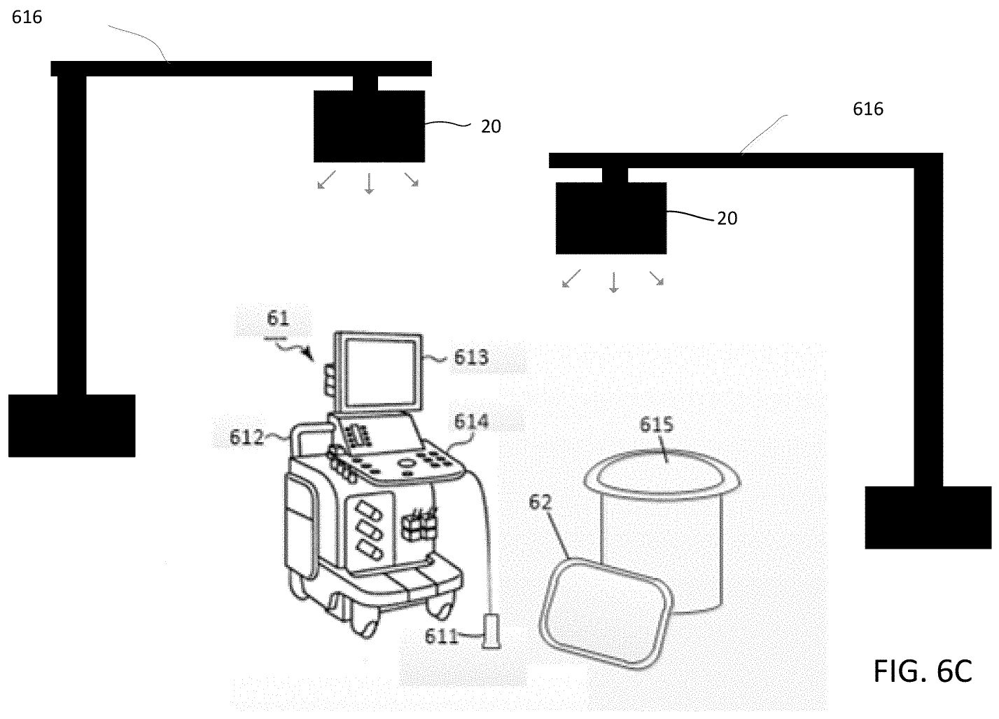

In one embodiment, the above mentioned techniques and structures can be applied to an ultrasound. FIG. 6A is a perspective view showing an example of the positional relationship between the apparatus main body 612 according to this embodiment, the ultrasound probe 611, the monitor 613, the input interface circuitry 614, the terminal equipment 62, the operator 63, and a patient P. A terminal equipment 62 may operate the ultrasound diagnostic apparatus 61.

In one embodiment, as shown in FIG. 6B, the ultrasound equipment may be placed inside of a cabinet or storage unit 617. The cabinet or storage unit 617 may have one or more UV light emitting sources 619, capable of emitting UV rays, placed at various locations within the cabinet or storage unit 617. Further, the cabinet or storage unit 617 may have doors to ensure that UV rays cannot escape. To disinfect the ultrasound equipment, it may be placed in the cabinet or storage unit 617 with the doors shut and the UV light emitting sources 619 on.

In one embodiment, as shown in FIG. 6C, one or more robotic arms 616 with UV light emitting sources 620 can be placed near the ultrasound equipment. When the robotic arms 16 and UV light emitting sources 620 are turned on, the UV light emitting sources 620 may emit UV rays, with the robotic arms 616 navigating the UV light emitting sources 620 to ensure that the ultrasound equipment has been disinfected (i.e. exposed to UV rays).

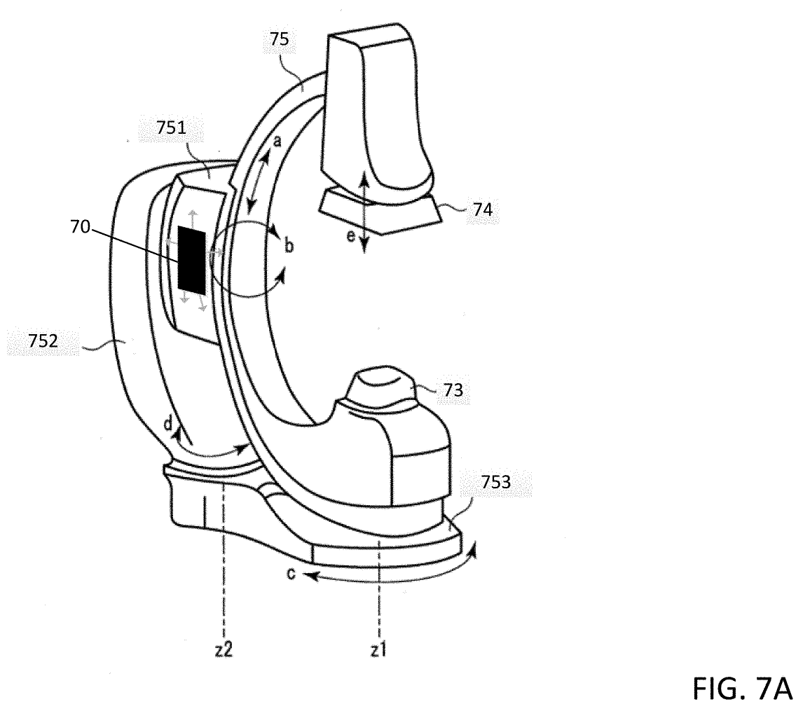

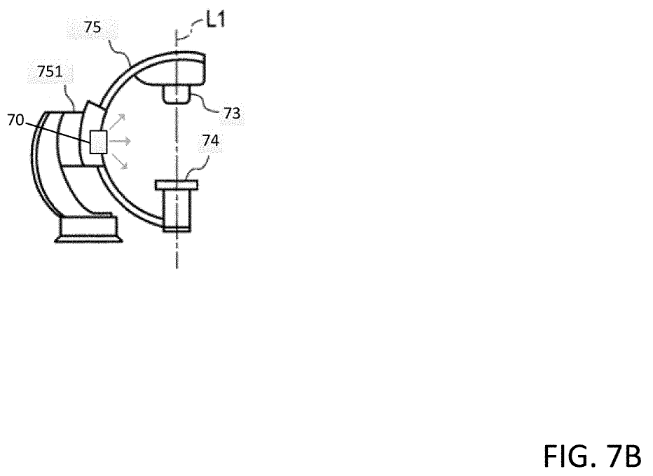

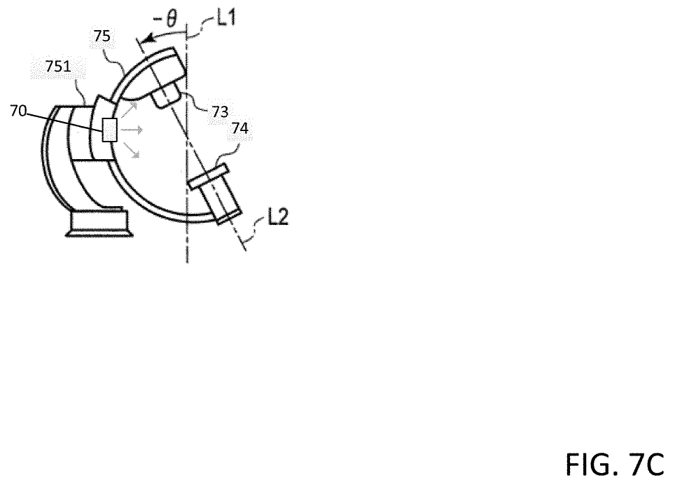

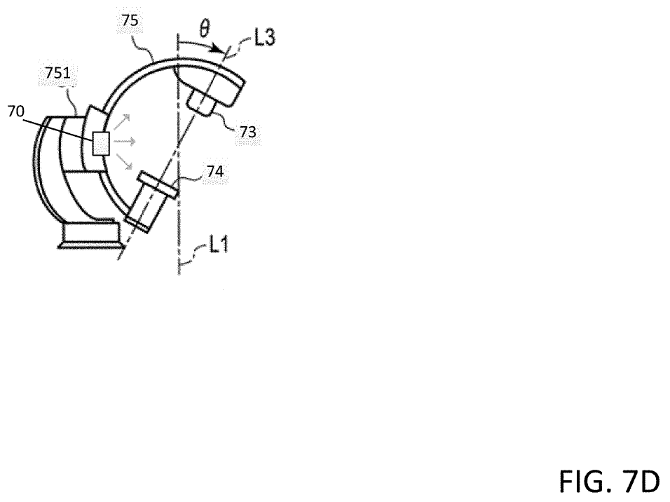

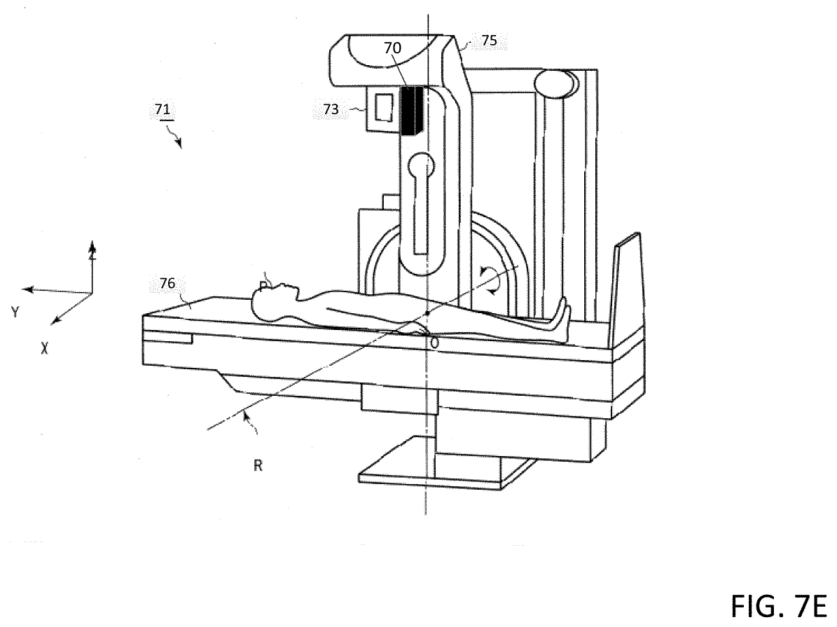

The above mentioned techniques and structures can be applied in an C-arm device (e.g., a C-arm X-ray device). FIG. 7A shows a non-limiting example of a C-arm X-ray device. In the C-arm device shown in FIG. 7A, a C-arm that includes the X-ray tube 73 and the X-ray detector 74 mounted on its ends is used as the supporter 75. The supporter 75 is supported by a stand 52 via a supporter holder 731. The supporter 75 is mounted on a side surface of the supporter holder 751 so as to be slidable in the directions of the arrow a. The supporter holder 751 is mounted so as to be rotatable in the directions of the arrow b with respect to the stand 752. The X-ray detector 74 is mounted so as to be slidable in the imaging axis direction (in the directions of the arrow e) connecting the X-ray tube 73 and the X-ray detector 74.

One end of a floor-turnable arm 753 arranged on a floor surface (not shown in the drawings) is mounted so as to be rotatable around a rotation axis z1 (in the directions of the arrow c) vertical to the floor surface. The other end of the floor-turnable arm 753 is mounted in such a manner that the stand 752 is rotatable around a rotation axis z2 (in the directions of the arrow d) vertical to the floor surface.

Furthermore, a UV light emitting source 70 can be attached to the C-arm device. This UV light emitting source 70 may emit UV rays in all directions when turned on to disinfect the C-arm device including the X-ray tube 73 and the X-ray detector 74.

FIG. 7B shows an example of a reference position of the C-arm device. FIG. 7B corresponds to a lateral view of the C-arm device shown in FIG. 7A. Moreover, FIG. 7B corresponds to the state in which the stand 752 shown in FIG. 7A is rotated in a direction of the arrow c by 90 degrees, and the supporter holder 51 shown in FIG. 7A is rotated by 180 degrees in a direction of the arrow b. Therefore, FIG. 7B shows a state in which the C-arm is rotated to position the X-ray tube 73 above the X-ray detector 74. At this time, the imaging axis L1 that connects the X-ray tube 73 and the X-ray detector 74 is vertical to the floor surface. The UV light emitting source 70 can be attached to the C-arm device and emit UV rays to disinfect various components of the C-arm device that are within the emission field of the light emitting source 70.

FIG. 7C shows an example in which the supporter 75 of the C-arm device is slid in a direction of the arrow a. Specifically, in FIG. 7C, the supporter 75 is slid in a direction that makes the X-ray tube 73 closer to the supporter holder 751, with reference to the C-arm device in the state shown in FIG. 7A. An imaging axis L2 that connects the X-ray tube 73 and the X-ray detector 74 after the sliding is inclined by an angle of −θ degrees with respect to the imaging axis L1. The UV light emitting source 70 is then closer to the X-ray tube 73 to facilitate disinfection of the X-ray tube 73.

FIG. 7D shows an example in which the supporter 75 of the C-arm device is slid in another direction of the arrow a. Specifically, in FIG. 7D, the supporter 75 is slid in a direction that takes the X-ray tube 73 away from the supporter holder 751, with reference to the C-arm device in the state shown in FIG. 7A. An imaging axis L3 that connects the X-ray tube 73 and the X-ray detector 74 after the sliding is inclined by an angle of θ degrees with respect to the imaging axis L1. The UV light emitting source 70 is then closer to the X-ray detector 74 to facilitate disinfection of the X-ray detector 74.

Furthermore, FIG. 7E shows an outer appearance of an X-ray diagnostic apparatus 71 in which a supporter 75 that supports an X-ray tube 73 is integrally formed with a bed that includes a table top 76. FIG. 7E corresponds to an X-ray fluoroscopic diagnosis apparatus typically used in gastrointestinal series examinations, etc. In FIG. 7E, a direction that passes through an effective focal spot of the X-ray tube 73 and that is vertical to a detection surface of an X-ray detector 74 (not shown) arranged below the table top 76 is defined as a z-axis, a direction that is along the long side of the table top 76 and that is vertical to the z-axis is defined as a y-axis, and a direction that is parallel to the z-axis and the y-axis (along the short side of the table top 76) is defined as an x-axis.

The supporter 75 supports the X-ray tribe 73 on one end, and the other end is supported by the bed. The bed supports the supporter 75. The supporter 75 rotates around a rotational axis R in the x axis direction, which passes through a fulcrum O. At this time, the attitude of the supporter 75 is defined by the angle by which the supporter 75 including the table top 76 rotates around the rotational axis R. The angle of the supporter 75 is determined in accordance with the inclination of the table top 76, with reference to the state of the table top 76 that is parallel to the floor surface, which is set as zero degrees.

A UV light emitting source 70 can be integrated to the X-ray diagnostic apparatus 71 so that various components of the X-ray diagnostic apparatus 71 (especially the table top 76) can be exposed to UV rays, and thus disinfected, when the UV light emitting source 70 is turned on. In one embodiment, the UV light emitting source 70 is movable relative to the table top 76 to disinfect the table top 76. In an alternate embodiment, the table top 76 is movable relative to the UV light emitting source 70 to disinfect the table top 76.

The function of the UV light emitting source(s) is to disinfect all and/or part of a medical imaging system. The UV light emitting source(s) can be placed in many possible locations throughout/nearby a medical imaging system, as illustrated through the various examples above. It should be understood that the illustrated examples are non-limiting, and the UV light emitting source(s) can be placed in many other potential locations so that its function can be achieved. For example, although illustrated as being robotic arms, in an alternate embodiment, mobile disinfectant towers may be used. Those towers may be moved manually or robotically including traversing along a track in the floor or using “line following” techniques (under the control of a controller) to automatically move the tower from one place (for disinfection) to another (for storage). The wheels, tracks, and/or lines and the corresponding controllers for driving the drive wheels to move the UV light emitting sources from location to location (e.g., along the track or line) are referred to herein as the transportation mechanism(s). The towers are placed proximate to the medical imaging equipment (i.e., sufficiently close to the medical imaging equipment so as to be able to disinfect the medical imaging equipment with UV light emitting source(s)).

In one embodiment, the medical imaging systems may include one or more status indicators. The status indicators could indicate whether or not a particular system has been disinfected, whether disinfecting is currently taking place, etc. Example of status indicators can be an LED light, notification to a computer system, voice prompt, etc.

In one embodiment, the UV light emitting source and/or robotic arm can be installed in or on the medical imaging equipment. Inn another embodiment, the UV light emitting source and/or robotic arm can be installed on the floor, or come out from underneath the floor. In another embodiment, the UV light emitting source and/or robotic arm can be installed to a wall, ceiling, or come out from within the walls or above the ceiling. In one embodiment, the UV light emitting sources and/or robotic arm are manufactured as part of the medical imaging equipment.

In one embodiment, the robotic arm can be preprogrammed to run specific functions, such as to disinfect specific areas and medical equipment after each procedure for specified areas of time. In another embodiment, the robotic arm can be controlled remotely by a human.

In an alternate embodiment, the medical imaging equipment discussed above is further configured to include a control system for selectively cleaning areas of the medical imaging equipment more intensely (e.g., for a longer duration or with in a higher-power cleaning mode) as compared with other areas. For example, according to one embodiment, the medical imaging equipment is equipped with at least one movable UV light source that passes over different areas of the medical imaging equipment. In one configuration, locations where a patient touches the medical imaging equipment or the structures around it (e.g., handles, tables, gantries) are monitored (e.g., (1) using black light rafter the patient has been imaged or (2) using at least one camera as the patient is interacting with and getting on and off the medical imaging equipment and generally shown in FIG. 4A), and the locations are associated with the procedure performed. For example, a particular handle is touched frequently when performing a standing type of imaging but a certain portion or portions of a table and a different support are touched when performing a prone type of image. Using those correlations, the disinfecting system is programmed in advance to clean the most touched or breathed on areas based on the type of procedure that was most recently performed.

In addition, characteristics of the patients can be used to further control the area to be programmed. For example, people of a certain, older age may utilize a support handle more than people of a certain younger age. Similarly, heavier people may touch more of an imaging table than lighter weight people. Those and similar factors can then be used to determine which portions of the medical imaging equipment need more disinfecting than others. In such a configuration, a camera or blacklight sensor is not needed at each location but rather the programming can be performed based on patient interactions using a test environment.

In a further embodiment, the medical imaging equipment is configured with sensors (generally shown in FIG. 4A) to determine patient interactions in real-time from data taken by actual patients. In such configurations, the blacklight sensor and/or the cameras do not use statistical information stored in advance but rather actually track a patient's interaction with the medical imaging equipment and adjust the disinfection routine using the information from the patient's actual interactions.

In either configuration (i.e., using stored information or using actual patient interaction information), the medical imaging system may further be equipped with sensors to detect whether addition disinfection is required. For example, acoustic sensors can be used to listen for the sounds of a patient coughing or sneezing, both of which would require additional disinfection (in terms or time, power level and an increased cleaning area). Additional sensors can be used as well, such as infrared sensors to (1) see where a patient warmed a particular area (indicating that the patient was close to the area) and/or (2) see if the patient was exhibiting elevated temperature and therefore might have been sick which requires more extensive cleaning. Humidity sensors similarly can be used to detect for wetness (e.g., caused by sneezing and/or perspiration) which may require additional disinfection.

In an embodiment where the UV light is built into the equipment, it can be turned on with a physical on/off switch on the side of the equipment. The UV on/off switch may also be operated by a separate remote control system. The UV system may self-decontaminate the equipment prior to, and/or subsequent to imaging exam of patient imaging. The UV impenetrable screen/curtain can be incorporated into the device, and the screen/curtain can be deployed simultaneously with turning on the UV light source.

One exemplary UV tower-based system that can be used in conjunction with the teachings herein is a multiple-emitter automated UV-C disinfection system by Surfacide (e.g., using 3 ultraviolet C (UV-C) emitters with a diameter of 59 cm and a height of 195 cm). The system utilizes a laser to identify the size of the disinfection space and to identify near-field objects. Based on these measurements the system then calculates the disinfection cycle time while rotating 360°.

Disclosed is medical imaging equipment with UV disinfection a system built into the imaging equipment. UV disinfection is a chemical-free process, and it leaves no residue behind post disinfection. Imaging systems with on board UV disinfection does not require additional system transportation and storage and/or handling of toxic or corrosive reagents/chemicals, which could also be detrimental to the imaging device. Other UV disinfection mechanisms require movement and/or multiple positioning UV equipment which add to workflow/time and decrease in productivity.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A disinfecting medical imaging system, comprising:
medical imaging equipment; and
a UV light source for disinfecting the medical imaging equipment.

2. The disinfecting medical imaging system as claimed in claim 1, wherein the medical imaging equipment comprises an X-ray imaging device.

3. The disinfecting medical imaging system as claimed in claim 1, wherein the medical imaging equipment comprises a Computed Tomography imaging device.

4. The disinfecting medical imaging system as claimed in claim 1, wherein the medical imaging equipment comprises a magnetic resonance imaging (MRI) imaging device.

5. The disinfecting medical imaging system as claimed in claim 1, wherein the medical imaging equipment comprises a Positron Emission Tomography (PET) imaging device.

6. The disinfecting medical imaging system as claimed in claim 1, wherein the UV light source comprises a composite UV light source.

7. The disinfecting medical imaging system as claimed in claim 1, wherein the UV light source comprises a UV-C light source.

8. The disinfecting medical imaging system as claimed in claim 1, further comprising a robotic arm for moving the UV light source into the medical imaging equipment.

9. The disinfecting medical imaging system as claimed in claim 1, further comprising a transportation mechanism for moving the UV light source proximate to the medical imaging equipment.

10. The disinfecting medical imaging system as claimed in claim 9, wherein the transportation mechanism comprises a track.

11. The disinfecting medical imaging system as claimed in claim 9, wherein the transportation mechanism comprises a line following controller.

12. The disinfecting medical imaging system as claimed in claim 1, wherein the medical imaging equipment comprises an ultrasound imaging device.

13. The disinfecting medical imaging system as claimed in claim 1, further comprising at least one of a cabinet and a storage device for housing the UV light source when disinfecting the ultrasound imaging device.

14. The disinfecting medical imaging system as claimed in claim 1, wherein the UV light source is integrated into the imaging equipment.

15. The disinfecting medical imaging system as claimed in claim 1, wherein the UV light source is attached to a C-arm of X-ray imaging equipment.

16. The disinfecting medical imaging system as claimed in claim 1, further comprising a table top, wherein the UV light source is moveably attached opposite to the table top to disinfect the table top.

17. The disinfecting medical imaging system as claimed in claim 1, further comprising a sensor for detecting areas that a patient interacted with in an imaging procedure.

18. The disinfecting medical imaging system as claimed in claim 17, wherein the sensor comprises a camera for detecting areas that the patient interacted with in the imaging procedure.

19. The disinfecting medical imaging system as claimed in claim 17, wherein the sensor comprises an acoustic sensor for detecting at least one of a cough and a sneeze.

20. The disinfecting medical imaging system as claimed in claim 17, wherein the sensor comprises a camera for detecting areas that the patient interacted with in an imaging procedure just preceding disinfecting.
